(19) United States
(12) Patent Application Publication
(10) Pub. No.: US 2026/0000592 A1
(43) Pub. Date: Jan. 1, 2026

(54) TREATMENT COMPOSITIONS WITH ESTERAMINES OR SALTS THEREOF

(71) Applicant: The Procter & Gamble Company, Cincinnati, OH (US)

(72) Inventors: William Peter MCDERMOTT, III, Cincinnati, OH (US); Laura ORLANDINI, LE MONT-SUR-LAUSANNE (CH); Sean N. NATOLI, Liberty Township, OH (US); Rajan Keshav PANANDIKER, West Chester, OH (US); Sophia Rosa EBERT, Mannheim (DE); Annalaura DEL REGNO, Mannheim (DE); Rafael TRUJILLO, Mason, OH (US)

(21) Appl. No.: 18/755,770

(22) Filed: Jun. 27, 2024

(51) Int. Cl. A61K 8/44; A61Q 13/00; C07C 227/18

(52) U.S. Cl. CPC A61K 8/44; A61Q 13/00; C07C 227/18

(57) ABSTRACT

A treatment composition that includes at least one perfume raw material and an esteramine or salt thereof, the esteramine or salt thereof may be obtained by a process of using catalytic amounts of at least one orthoester; and methods of treating an article or a surface, wherein the method comprises treating the article or surface with such treatment composition, optionally in the presence of water.

### FIELD OF THE INVENTION

The present disclosure relates to treatment compositions that include at least one perfume raw material and esteramines or salts thereof; and methods of treating an article or surface by using such compositions. The esteramines may be obtained with a process that employs a catalytic or a sub-stoichiometric amount of an orthoester.

### BACKGROUND OF THE INVENTION

Consumers prefer treatment products (e.g., fabric treatment compositions, hard surface treatment compositions, etc.) that have a noticeably pleasant smell. The consumer products industry is continuously seeking ways to improve the delivery efficiency and/or performance benefits associated with the perfume raw materials contained in such treatment compositions. The use of perfume delivery systems (e.g., a pro-perfume) can be a useful strategy to improve delivery efficiency. Accordingly, there is continued interest in new and improved perfume delivery systems for use in treatment compositions.

Moreover, due to increasing environmental concerns, consumers want products that are associated with desirable environmental or sustainability profiles. Accordingly, some important targets of the consumer products industry are to utilize more biodegradable ingredients, improve the sustainability of the cleaning formulations, avoid the accumulation of non-degradable compounds in the ecosystem, and lower CO₂ emissions associated with making or using the consumer product. Hence, there is a continuing need to provide consumer products that are more environmentally friendly but don't sacrifice performance.

In combination, there is a continuing need for treatment compositions that include perfume delivery systems that enable good/increased perfume delivery performance along with possessing increased environmentally friendliness. There is also a continuing need for methods of treating an article or surface by using such treatment compositions.

### SUMMARY OF THE INVENTION

The present disclosure relates to treatment compositions that include at least one perfume raw material and an esteramine of Formula (I) or salt thereof,

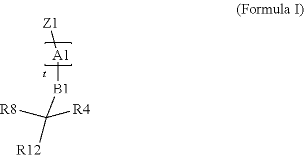

wherein independently from each other:
t being an integer from 1 to 100;
A₁ is independently for each repetition unit t selected from the list consisting of ethyleneoxy group, 1,2-propyleneoxy group, 1,2-butyleneoxy group, 2,3-butyleneoxy group, i-butyleneoxy group, pentyleneoxy group, hexyleneoxy group, styryloxy group, decenyloxy group, dodecenyloxy group, tetradecenyloxy group, and hexadecanyloxy group, wherein for t equal to 1 the oxygen atom of the A₁ group is bound to the B group and the following A₁ group is always bound via the oxygen atom to the previous A₁ group;
B₁ is independently from each other selected from the group consisting of a bond, linear C₁ to C₁₂ alkanediyl groups, and branched C₁ to C₁₂ alkanediyl groups;
R₄, R₈, and R₁₂ being selected from the group consisting of H, linear alkyl, branched alkyl, and cycloalkyl;
with the provisio that Z₁ is selected from the group consisting of alanine, arginine, asparagine, aspartic acid, cysteine, glutamine, glycine, histidine, isoleucine, leucine, lysine, methionine, phenylalanine, proline, serine, threonine, tryptophan, tyrosine, valine, and a compound according to Formula (II), wherein said compound according to Formula (II) connects to the compound according to Formula (I) via the bond labeled with *, with the provisio of at least one group R₄, R₈, and/or R₁₂ containing at least 7 or more carbon atoms;

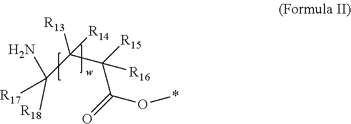

with independently from each other
w being an integer from 0 to 12;
R₁₃ and R₁₄ independently for each repetition unit w being selected from the group consisting of H, linear alkyl, branched alkyl, and cycloalkyl;
R₁₅, R₁₆, R₁₇, and R₁₈ being selected from the group consisting of H, linear alkyl, branched alkyl, and cycloalkyl.

The present disclosure also relates to treatment compositions that include at least one perfume raw material and an esteramine or salt thereof, the esteramine or salt thereof obtained by a process of using catalytic amounts of at least one orthoester, the process comprising the steps of: a reaction of: 1) at least one amino acid selected from alpha-, beta-, gamma-, delta-, epsilon-etc. amino acids, such as alanine, glycine, leucine, isoleucine, valine, proline, phenylalanine, arginine, asparagine, aspartic acid, aspartate, glutamine, glutamate, histidine, lysine, threonine, tryptophan, tyrosine, cysteine, methionine, serine; alpha-amino acids with secondary or tertiary amino groups such as sarcosine, N,N-dimethylglycine; other amino acids such as 6-aminohexanoic acid, 4-aminobutanoic acid, 3-aminopropanoic acid, 12-aminododecanoic acid, 11-aminoundecanoic acid; amino acids formally derived from the hydrolysis of α-lactam (three ring atoms), β-lactam (four ring atoms), γ-lactam (five ring atoms) and so on; such lactams preferably being β-propiolactam, g-butyrolactam, δ-valerolactam, g-valerolactam, e-caprolactam, d-decalactam, g-decalactam, e-decalactam; preferably alanine, valine, beta-alanine, 6-aminohexanoic acid; with 2) at least one alcohol (A) bearing at least one hydroxy group, being selected from mono-, di- and polyols, all of which may be optionally alkoxylated, wherein the alkoxylation of the at least one hydroxy group takes place in a step before step a), with the alcohol being alkoxylated with at least one alkylene oxide, preferably at least 1 and up to 200, preferably 1 to 100, more preferably up to 50 moles alkylene oxide per hydroxy group; in the presence of 3) at least one acid (C), being selected from inorganic and organic acids, wherein said organic or inorganic acid has preferably a pKa value in the range of from −3 and up to +5, more preferably from −2.5 to 1.5, preferably at least one organic acid, such as sulfonic acids, more preferably alkylsulfonic acid and/or arylsulfonic acid; and 4) in the presence of at least one orthoester, such as triethyl orthoformate, trimethyl orthoformate, triethyl orthoacetate, trimethyl orthoacetate, and the like; whereas the orthoester is used in sub-stoichiometric amounts, preferably catalytic amounts (referred to the amino acid), and whereas the alcohol used for the esterification is different from the alcohol-residual in the orthoester; to produce an esteramine salt. Optionally, there can be an additional step of neutralization of the obtained esteramine salt with at least one base to obtain the free esteramine. The treatment composition includes, by weight of the treatment composition, less than 0.5% of alcohol (A) as a residual of the reaction process which obtained the esteramine or salt thereof.

The present disclosure also relates to a method of treating an article or a surface, wherein the method includes treating the article or surface with the treatment composition as detailed herein, optionally in the presence of water.

### DETAILED DESCRIPTION OF THE INVENTION

The present disclosure relates to treatment compositions that include at least one perfume raw material and esteramines or salts thereof; as well as methods of treating an article or surface by using such compositions. In some embodiments of treatment compositions, some of the at least one perfume raw material may join to the esteramine or salt thereof by linking bond at some point (e.g., when added together as a premix, when added together in the consumer product bottle, when contacted with an article or surface, in the scrubbing portion of a hard surface cleaning process, in the wash process of fabric laundering, in the drying process, etc.). Without wishing to be bound by theory, it is believed that when the linking bond later breaks (e.g., via hydrolysis or reversion), the perfume raw material is released and delivers a perfume delivery benefit.

Such treatment compositions, and related methods of use, are described in more detail below. Such treatment compositions utilize esteramines and salts thereof that are obtained by the processes detailed herein. Treatment compositions that employ the esteramines and salts thereof that are obtained by the reaction processes detailed herein will have less residual fatty alcohols (i.e., alcohol (A) as defined herein), when compared with esteramines and salts thereof that are obtained by different, known reaction processes (e.g., esteramine synthesized with no such orthoester). Accordingly, due to having less residual fatty alcohols in the treatment composition, the treatment composition will have a better base odor. This can be confirmed through GC/MS (gas chromatography/mass spectrometry) headspace analysis comparing treatment compositions that only vary in the source of the esteramines and salts thereof, and that source varies by the reaction process in which the esteramines and salts thereof are derived.

Further, for stability reasons, the processes detailed herein may preferably employ mono and secondary alcohols for the reactant that is the at least one alcohol (A). Employing mono and secondary alcohols for the reactant that is the at least one alcohol (A) will also lead to the treatment composition containing residual mono and secondary alcohols that were not converted into esteramines and salts thereof. Esteramines obtained by mono and secondary alcohols have improved stability in comparison to other esteramines.

Obviously the esteramines and salts thereof obtained by the processes detailed herein (and esteramines and salts thereof obtained by known processes using stoichiometric amounts of orthoesters) can be further refined/purified to remove even more residual fatty alcohols, and therefore improve the base odors of the treatment compositions in which they are employed. However, each refining/purification step uses more energy and makes the resulting consumer product less environmentally friendly. Accordingly, the ability to employ the esteramines and salts thereof obtained from the processes defined herein—without additional refining steps—in the treatment compositions detailed herein, allows for an acceptable, more environmentally friendly treatment composition. In other words, such treatment compositions that utilize esteramines that are obtained by the processes detailed herein also have a lower CO₂ footprint when compared to esteramines made through alternate processes. In addition to the benefits noted above, such esteramines and salts thereof show good biodegradability properties when being used in treatment compositions, for example, fabric treatment compositions.

### Definitions

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

Specifically, the term “free of water” means that the composition contains no more than 5 wt.-% of water based on the total amount of solvent, in another embodiment no more than 1 wt.-% of water based on the total amount of solvent, in a further embodiment the solvent contains no water at all.

Generally, as used herein, the term “obtainable by” means that corresponding products do not necessarily have to be produced (i.e., obtained) by the corresponding method or process described in the respective specific context, but also products are comprised which exhibit all features of a product produced (obtained) by said corresponding method or process, wherein said products were actually not produced (obtained) by such method or process. However, the term “obtainable by” also comprises the more limiting term “obtained by”, i.e., products which were actually produced (obtained) by a method or process described in the respective specific context.

When used herein any definition requiring a compound or a substituent of a compound to consist of “at least [a number] of carbon atoms”, that number of carbon atoms refers to the total number of carbon atoms in said compound or substituent of a compound. For example, for a substituent disclosed as “alkyl ether with at least 8 carbon atoms comprising alkylene oxide groups”, the total number of at least 8 carbon atoms needs to be the sum of the number of carbon atoms of the alkyl moiety and the number of carbon atoms of the alkylene oxide moieties.

All such terms not specifically defined have their ordinary meaning as known in the field of organic chemistry.

The term “containing one hydroxy group” means that only one group-OH is present. Any functionalized group derived from a hydroxy group such as an ether group is not considered to be an —OH group.

The term “containing at least two hydroxy groups” means that two or more-OH groups are present. The term “hydroxy group” is equal to the term “hydroxyl group” or “—OH group”. Alcohols/compounds having only one hydroxy group, such as methanol or ethanol, do, by consequence, not fall under the definition of an alcohol containing at least two hydroxy groups according to compound (A) of the present disclosure. Any functionalized group derived from a hydroxy group such as an ether group is not considered to be an —OH group.

“Sulfonates” in this present disclosure are the anions derived from sulfonic acids, preferably alkylsulfonic acid and/or arylsulfonic acid, more preferably alkylsulfonic acid, most preferably methanesulfonic acid; such acids are used to at least partially protonate the esteramines, thus forming the sulfonates of the esteramines.

As used herein, the articles “a” and “an” when used in a claim or an embodiment, are understood to mean one or more of what is claimed or described. As used herein, the terms “include(s)” and “including” are meant to be non-limiting, and thus encompass more than the specific item mentioned after those words.

The term “about” as used herein encompasses the exact number “X” mentioned as e.g. “about X %” etc., and small variations of X, including from minus 5 to plus 5% deviation from X (with X for this calculation set to 100%), preferably from minus 2 to plus 2%, more preferably from minus 1 to plus 1%, even more preferably from minus 0.5 to plus 0.5% and smaller variations. Of course, if the value X given itself is already “100%” (such as for purity etc.) then the term “about” clearly can and thus does only mean deviations thereof which are smaller than “100”.

As used herein, “treatment compositions” includes consumer product compositions utilized in treating one or more surfaces. The treatment compositions of the present disclosure may be useful in baby care, beauty care, fabric care, home care, family care, feminine care, and/or health care applications, and may be used to treat, for example, hard surfaces, soft surfaces, fabric, hair, and/or or skin. For clarity, the term “treatment composition” is an umbrella term that includes the non-limiting examples of “fabric treatment compositions”, “fabric care compositions”, and “hard surface treatment compositions.”

As used herein the phrase “fabric care composition” includes compositions and formulations designed for treating fabric. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (° C.) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Structure of Esteramines and Salts Thereof:

The treatment compositions detailed herein relate to alkoxylated esteramines of Formula (I) and salts thereof,

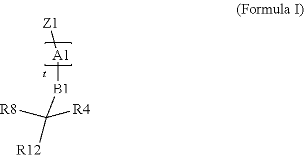

wherein independently from each other:
t being an integer from 1 to 100;
A₁ is independently for each repetition unit t selected from the list consisting of ethyleneoxy group, 1,2-propyleneoxy group, 1,2-butyleneoxy group, 2,3-butyleneoxy group, i-butyleneoxy group, pentyleneoxy group, hexyleneoxy group, styryloxy group, decenyloxy group, dodecenyloxy group, tetradecenyloxy group, and hexadecanyloxy group, wherein for t equal to 1 the oxygen atom of the A₁ group is bound to the B group and the following A₁ group is always bound via the oxygen atom to the previous A₁ group.
B₁ is independently selected from the group consisting of a bond, linear C₁ to C₁₂ alkanediyl groups, and branched C₁ to C₁₂ alkanediyl groups;
R₄, R₈, and R₁₂ being selected from the group consisting of H, linear alkyl, branched alkyl, and cycloalkyl;
with the provisio that Z₁ is selected from the group consisting of alanine, arginine, asparagine, aspartic acid, cysteine, glutamine, glycine, histidine, isoleucine, leucine, lysine, methionine, phenylalanine, proline, serine, threonine, tryptophan, tyrosine, valine, and a compound according to Formula (II), wherein said compound according to Formula (II) connects to the compound according to Formula (I) via the bond labeled with *, with the provisio of at least one group R₄, R₈, and/or R₁₂ containing at least 7 or more carbon atoms;

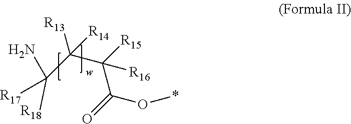

with independently from each other
w being an integer from 0 to 12;
R₁₃ and R₁₄ independently for each repetition unit w being selected from the group consisting of H, linear alkyl, branched alkyl, and cycloalkyl;
R₁₅, R₁₆, R₁₇, and R₁₈ being selected from the group consisting of H, linear alkyl, branched alkyl, and cycloalkyl.

Esteramines and salts thereof according to the present disclosure may be employed in treatment compositions, in particular, liquid fabric enhancers. In some embodiments of treatment compositions, some of the at least one perfume raw material may join to the esteramine or salt thereof by linking bond. Without wishing to be bound by theory, it is believed that when the linking bond later breaks (e.g., via hydrolysis or reversion), the perfume raw material is released and delivers a perfume delivery benefit. Accordingly, the esteramines and salts thereof as described herein may be used as part of a pro-perfume technology.

In the following, the various embodiments of the present disclosure are described in more detail:
A₁ is independently for each repetition unit t selected from the list consisting of ethyleneoxy group, 1,2-propyleneoxy group, 1,2-butyleneoxy group, 2,3-butyleneoxy group, i-butyleneoxy group pentenyloxy group, hexyloxy group, styryloxy group, decenyloxy group, dodecyloxy group, tetradecenyloxy group and hexadecenyloxy group, wherein for t equal to 1 the oxygen atom of the A₁ group is bound to the B group and the following A₁ groups are always bound via the oxygen atom to the previous A₁ group. When t is equal to or more than 2, the independently selected A₁ either form a randomly distributed sidechain of various alkylenyloxy units or the form a block structure with at least one alkylenyloxy group repeating itself at least two times, optionally followed by further blocks of different alkylenyloxy group repeating themselves at least two times.

In one embodiment A₁ is independently for each repetition unit t selected from the list consisting of ethylenoxy group, 1,2-propyleneoxy group1,2-1,2-propyleneoxy group, and 1,2-butylenoxy group. In another embodiment, A₁ forms a block of at least two ethyleneoxy groups followed by a block of at least two propylenoxy groups, optionally followed by another block of at least two ethyleneoxy groups. In another embodiment, A₁ forms a block of at least two 1,2-propyleneoxy groups followed by a block of at least two ethylenoxy groups, optionally followed by another block of at least two 1,2-propyleneoxy groups. In another embodiment, A₁ is selected from the list consisting of ethyleneoxy group, 1,2-propyleneoxy group, and 1,2-butyleneoxy group in such a way that at least one block of ethyleneoxy groups, 1,2-propyleneoxy groups, or 1,2-butyleneoxy groups is formed, optionally followed by one or more blocks of ethyleneoxy groups, 1,2-propyleneoxy groups, or 1,2-butyleneoxy groups. In another embodiment, A₁ is ethyleneoxy groups. In another embodiment, A₁ is 1,2-propyleneoxy groups. In another embodiment, A₁ is selected in such a way that a block of one to five ethyleneoxy groups is followed by a block of one to three propylenoxy groups followed by a block of one to five ethylenoxy groups.

In one embodiment t is in the range of from 1 to 30. In another embodiment t is in the range of from 1 to 20. In another embodiment t is in the range of from 2 to 10.

In one embodiment of the present disclosure, B₁ is selected from the group consisting of a bond, and linear C₁ to C₁₂ alkanediyl groups. In another embodiment, B₁ is selected from the group consisting of a bond, and linear C₁ to C₆ alkanediyl groups. In another embodiment, B₁ is selected from the group consisting of a bond, and linear C₁ to C₃ alkanediyl groups. In another embodiment, B₁ is selected from the group consisting of a bond, and a C₁ alkanediyl group. In another embodiment B₁ is selected from the group consisting of a bond, and a C₁ alkanediyl group. In another embodiment B₁ is bond.

In one embodiment of the present disclosure, R₄, R₈, and R₁₂ are all independently selected from the group consisting of H, linear alkyl, branched alkyl, and cycloalkyl. In one embodiment, R₄, R₈, and R₁₂ are all independently selected from the group consisting of H, linear C₁ to C₁₂ alkyl, and C₁ to C₁₂ branched alkyl. In another embodiment, R₄, R₈, and R₁₂ are all independently selected from the group consisting of H, linear C₁ to C₆ alkyl, and C₁ to C₉ branched alkyl.

For Z₁ being selected a compound according to Formula (II), said compound according to Formula (II) connects to the compound of Formula (I) via the bond labeled with *,

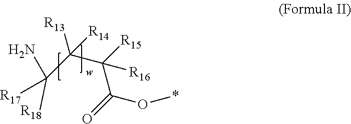

with independently from each other
w being an integer from 0 to 12;
R₁₃ and R₁₄ independently for each repetition unit w being selected from the group consisting of H, linear alkyl, branched alkyl, and cycloalkyl;
R₁₅, R₁₆, R₁₇, and R₁₈ being selected from the group consisting of H, linear alkyl, branched alkyl, and cycloalkyl. In one embodiment of the present invention, R₁₃, R₁₄, R₁₅, R₁₆, R₁₇, and R₁₈ are all independently selected from the group consisting of H, linear C₁ to C₁₂ alkyl, and C₁ to C₁₂ branched alkyl. In another embodiment, R₁₃, R₁₄, R₁₅, R₁₆, R₁₇, and R₁₈ are all independently selected from the group consisting of H, linear C₁ to C₆ alkyl, and C₁ to C₉ branched alkyl.

In one embodiment of the present disclosure Z₁ is selected from the group consisting of alanine, glycine, lysine, and of compounds according to Formula (II), wherein w is an integer in the range of from 1 to 4, and the compound according to Formula (II) connects to the compound according to Formula (I) via the bond labeled with *, with the provisio of at least one group R₄, R₈, and/or R₁₂ containing at least 7 or more carbon atom. In another embodiment Z₁ is alanine. In another embodiment Z₁ is a compound according to Formula (II) with w=0 and R₁₅ to R₁₈ are all H. In another embodiment Z₁ is a compound according to Formula (II) with w=1 and R₁₃ to R₁₈ are all H. In another embodiment Z₁ is a compound according to Formula (II) with w=3 and R₁₃ to R₁₈ are all H.

In another embodiment of the present disclosure, B₁ is selected from branched or linear C₁- to C₁₂-alkyl and R₈ is selected from linear or branched C₆- to C₂₃-alkyl. In another embodiment of the present invention B₁ is selected from branched or linear C₁- to C₁₂-alkyl and R₈ is selected from linear or branched C₁- to C₃-alkyl. Another embodiment consists of B₁ being 2-ethyl-ethandiyl and R₈ being linear C₃-alkyl.

In another embodiment of the present disclosure B₁ is selected from branched or linear C₁- to C₁₂-alkyl and R₈ is selected from linear or branched C₁- to C₃-alkyl, and Z₁ is a is a compound according to Formula (II) with w=3 and R₁₃ to R₁₈ are all H.

In another embodiment of the present disclosure B₁ is selected from branched or linear C₆- to C₁₂-alkyl and R₈ is selected from linear or branched C₁- to C₃-alkyl, t is in the range of from 1 to 10, A₁ is for each repetition unit t ethyleneoxy group, and Z₁ is selected from the group consisting of alanine, a compound according to Formula (II) with w=0 and R₁₅ to R₁₈ all H, a compound according to Formula (II) with w=1 and R₁₃ to R₁₈ all H, and a compound according to Formula (II) with w=3 and R₁₃ to R₁₈ all H.

In another embodiment of the present disclosure B₁ is selected from branched or linear C₆- to C₁₂-alkyl and R₈ is selected from linear or branched C₁- to C₃-alkyl, R₄ and R₁₂ are selected from H and linear or branched C₁- to C₃-alkyl, t is in the range of from 1 to 10, A₁ is for each repetition unit t 1,2-propyleneoxy group, and Z₁ is selected from the group consisting of alanine, a compound according to Formula (II) with w=0 and R₁₅ to R₁₈ all H, a compound according to Formula (II) with w=1 and R₁₃ to R₁₈ all H, and a compound according to Formula (II) with w=3 and R₁₃ to R₁₈ all H.

The esteramines according to the present disclosure are obtained either as free amines, as salts thereof or as a mixture of free amines and salts. Salts are formed by at least partial protonation of the amine groups by an acid being a protic organic acid or a protic inorganic acid. In one embodiment, the acid for at least partial protonation of the amine groups is selected from the group consisting of methanesulfonic acid, hydrochloric acid, hydrobromic acid, sulfuric acid, phosphoric acid, citric acid, and lactic acid. In one embodiment, the acid is selected from the group of methanesulfonic acid, hydrochloric acid, and sulfuric acid. In another embodiment, the acid is methanesulfonic acid.

Partial protonation in one embodiment is protonation of the amine groups in the range of from 1 to 99 mol-% of all amine groups, in another embodiment in the range of from 10 to 90 mol-% of all amine groups, in another embodiment in the range of from 25 to 85 mol-%, in another embodiment in the range of from 40 to 75 mol-% of all amine groups.

### Processes to Produce Esteramines and Salts Thereof:

It has been surprisingly found that the esterification of amino acids can be improved by use of sub-stoichiometric amounts and even catalytic amounts of orthoesters. Further and unexpectedly, esteramines can be produced wherein the ester is derived from another alcohol than that present in the orthoester which is used as catalyst. Thus, the present disclosure relates to an improved process of producing esteramines and their salts using sub-stoichiometric or even catalytic amounts of orthoesters.

Hence, the present disclosure relates to treatment compositions that comprise esteramines or salts thereof that are produced by using catalytic amounts of at least one orthoester, the process comprising the steps of:
a) reacting:
   i) at least one amino acid selected from alpha-, beta-, gamma-, delta-, epsilon-etc. amino acids, such as alanine, glycine, leucine, isoleucine, valine, proline, phenylalanine, arginine, asparagine, aspartic acid, aspartate, glutamine, glutamate, histidine, lysine, threonine, tryptophan, tyrosine, cysteine, methionine, serine; alpha-amino acids with secondary or tertiary amino groups such as sarcosine, N,N-dimethylglycine; other amino acids such as 6-aminohexanoic acid, 4-aminobutanoic acid, 3-aminopropanoic acid, 12-aminododecanoic acid, 11-aminoundecanoic acid; amino acids formally derived from the hydrolysis of α-lactam (three ring atoms), β-lactam (four ring atoms), γ-lactam (five ring atoms) and so on; such lactams preferably being β-propiolactam, g-butyrolactam, δ-valerolactam, g-valerolactam, e-caprolactam, d-decalactam, g-decalactam, e-decalactam; preferably alanine, valine, beta-alanine, 6-aminohexanoic acid;
   with:
   ii) at least one alcohol bearing at least one hydroxy group, being selected from mono-, di- and polyols, all of which may be optionally alkoxylated, wherein the alkoxylation of the at least one hydroxy group takes place in a step before step a), with the alcohol being alkoxylated with at least one alkylene oxide, preferably at least 1 and up to 200, preferably 1 to 100, more preferably up to 50 moles alkylene oxide per hydroxy group; in presence of:
   iii) at least one acid (C), being selected from inorganic and organic acids, wherein said organic or inorganic acid has preferably a pKa value in the range of from −3 and up to +5, more preferably from −2.5 to 1.5, preferably at least one organic acid, such as sulfonic acids, more preferably alkylsulfonic acid and/or arylsulfonic acid, more preferably alkylsulfonic acid, most preferably methanesulfonic acid;
   and:
   iv) in the presence of at least one orthoester, such as triethyl orthoformate, trimethyl orthoformate, triethyl orthoacetate, trimethyl orthoacetate, and the like;
   wherein the orthoester is used in sub-stoichiometric amounts, preferably catalytic amounts (referred to the amounts of the amino acid), and
wherein the alcohol used for the esterification is different from the alcohol-residual in the orthoester;
to produce an esteramine salt;
b) optionally remove water and/or excess alcohol during and/or after the reaction to obtain the purified esteramine salt;
c) optionally neutralize the obtained esteramine salt with at least one base to obtain the free esteramine.

Further, the following process steps can be optionally included in the process disclosed herein:

The reaction mixture may be inertized before and/or during the reaction, preferably prior to the addition of the acid C. Inertization can be achieved by a steam of gas such as nitrogen or argon, and/or by adding an oxygen-scavenging compound. Such oxygen-scavengers are known. Advantageous is the use of small amounts of inorganic salts with reducing properties towards oxygen, such as a hypophosphorous compound, such as hypophosphorous acid or any of their salts, especially any alkali or ammonium or earth alkali salts, more preferably the acid, which can be added to the reaction mixture prior to adding the sulfuric acid. As one of the effects, this addition of the oxygen-scavenging compound leads to an improved color of the resulting product, i.e., meaning a reduced coloring, when compared to omitting such addition.

The reaction is performed the reaction is performed:
at a temperature of from 50 to 200° C., preferably 70-180° C., more preferably 80° C.-160° C., most preferably 120° C.-150° C., such as 60, 65, 75, 85, 90, 95, 100, 110, 115, 120, 125, 130, 135, 140, 145, 155, 165, 170, 190° C.;
for a period of from 1 to 30, preferably from 2, more preferably from 3 hours, even more preferably at least 5 hours, and preferably up to 48, more preferably up to 20, even more preferably up to 15 hours, such as preferably 3 to 24, more preferably 5 to 24, most preferably 10-24 hour(s); and
at from 0.001 to 10 bar pressure, such as from 0.001, more preferably from 0,005, even more preferably from 0.1, and preferably up to 8, more preferably up to 5, even more preferably up to 4 bar, such as 1 to 10, more preferably 1 to 5, even more preferably 1 to 4 bar, or such as 1 to 1000 mbar, more preferably 100 to 500 mbar.

Preferably, the temperature, the duration and the pressure are both as defined individually before, preferably the preferred ranges of all three parameters, more preferably the more preferred ranges and so on of all three parameters are selected. In one embodiment, the temperature is kept constant for the duration of the reaction. In another embodiment, the temperature is varied within the temperature range during the duration of the reaction.

In one embodiment disclosed herein, the reaction with acid C is carried out under atmospheric pressure. In another embodiment the reaction with acid C is carried out in a closed vessel under a pressure of from 0.001 to 1 bar. In another embodiment the reaction with acid Cis carried out in a closed vessel under pressure of more than 1 to 10 bar.

In one embodiment a protective atmosphere of an inert gas such as nitrogen gas or argon gas is used to carry out the reaction.

In another embodiment inertization is carried out by adding an oxygen-scavenging agent, preferably small amounts of inorganic salts with reducing properties towards oxygen, such as a hypophosphorous compound, such as hypophosphorous acid or any of their salts, especially any alkali or ammonium or earth alkali salts, more preferably the acid, a hypophosphorous compound, more preferably hypophosphorous acid. In another embodiment inertization is carried out by adding an oxygen-scavenging agent as defined before and with the use of a protective atmosphere of an inert gas as defined before.

It is clear that the temperature, pressure, and duration can be chosen independently from the disclosed values and ranges and combined with each other. Similarly, also the way inertization is achieved can be chosen and combined with the temperatures, durations and pressures chosen, all as defined before.

During or following the reaction, preferably at least during the reaction, water and/or excess alcohol can be removed. Removal of water and alcohol can be carried out by all techniques known in the art, for example by application of a stream of gas and/or applying a distillation method, preferably a distillation, more preferably a distillation method under reduced pressure and/or at elevated temperature, preferably both, a preferred method being the use of an apparatus such as a Dean-Stark-trap.

In another embodiment, water and/or alcohol is removed using a stream of gas, such as using gas such as inert gas as nitrogen or argon, preferably nitrogen, or steam made from water, preferably using inert gas, more preferably nitrogen. In a more preferred embodiment, water and/or alcohol is removed by application of a vacuum and/or increasing the temperature, most preferably using an apparatus such as a Dean-Stark-trap. The optional removal of water and/or excess of alcohol is more preferably carried out applying a vacuum in the range of from 0.1 mbar to 800 mbar, preferably of from 1 mbar to 500 mbar and more preferably of from 10 mbar to 100 mbar and using elevated temperatures.

In a preferred embodiment, during the reaction a steam of inert gas, such as nitrogen or argon, preferably nitrogen, is passed through the reaction mixture. This helps to keep the reaction mixture inert, but also supports the removal of forming water from the chemical reaction, and thus to control the amount of water present.

The molar ratio of amino acid to hydroxyl group of the (optionally alkoxylated) alcohol is:
(0.8*n):1 to (1*n):1.5, with the number of hydroxy groups of the (optionally alkoxylated) alcohol being n.

In one embodiment, the process is carried out with the molar ratio of the acid C to the amino acid being in the range of from 0.8:1 to 1.2:1.

Suitable solvents for the reaction may be toluene, xylene, heptanol, cyclohexene, and the like, preferably the reaction is carried out without solvent.

In a preferred embodiment, the reaction is performed with the minimum practical amount of water being present, preferably substantially free of water, and most preferably free from water during the start and the most part of the reaction, more preferably from start to essentially complete reaction

Obviously, such amino acids are known, and can be prepared usually from natural sources. Others can be produced starting from natural sources by modifying them to obtain amino acids as starting material. If such modification does result in the addition of carbon-atoms, e.g., by chemical addition reactions, and if such addition reactions use non-fossil carbon atoms, then amino acids contain only non-fossil-based carbon. Hence, it is preferred, that only naturally occurring amino acids and/or nature-derived amino acids (not being made with the addition of non-fossil carbon in building blocks to obtain such nature-derived amino acids) are employed in the process disclosed herein. Even more preferably, only naturally occurring amino acids are employed.

The same applies for the lactams: whenever a lactam is accessible from natural sources, such natural or nature-derived lactam is preferred; more preferred are of course naturally occurring lactams, to increase the overall content in non-fossil-based carbon content.

The at least one alcohol bearing at least one hydroxy group, being selected from mono-, di- and polyols, all of which may be optionally alkoxylated, wherein the alkoxylation of the at least one hydroxy group takes place in a step before step a), with the alcohol being alkoxylated with at least one alkylene oxide, preferably at least 1 and up to 200, preferably 1 to 100, more preferably up to 50 moles alkylene oxide per hydroxy group, and in one embodiment even more preferably up to 20, such as up to 15 or even up to 10 moles alkylene oxide per hydroxy group.

Alcohols containing one hydroxy group are well-known in the art. Similarly, alcohols containing at least two hydroxy groups according to compound (A) are known to a person skilled in the art. As mentioned above, the respective alcohol may contain one, two, three, four, five or even more hydroxy groups within the respective molecule/compound. The respective alcohol may contain linear, branched and/or cyclic alkyl fragments. Beyond that, the respective alcohol may also contain aromatic fragments as well as combinations of alkyl and aromatic fragments (“aralkyl” fragments). Furthermore, the respective alcohol may also contain alkyl ether fragments. Examples of alcohols according to compound (A) are glycerol, pentaerythritol, sorbitol, 1,1,1-trimethylolpropane (TMP), erythritol or alkoxylated alcohols, such as polyethylene glycol.

Many alcohols according to compound (A) of the present disclosure are commercially available, for example, under the tradename “Pluronic(s)” or “Pluriol” (for example as polyethylene glycol block (co) polymers) from BASF SE.

The alcohol (A) for the inventive esteramine and the inventive process as described herein is selected from:
(a) mono-alcohols such as C1- to C36-alkanols, selected from the groups non-alkoxylated linear
   C2- to C36-alcohols, such as mixture of such alcohols selected from C6- to C22-fatty alcohols, preferably C8- to C22-fatty alcohols, more preferably C12- and C14-fatty alcohols, most preferably C16- and C18-fatty alcohols; non-alkoxylated branched C3-to C36-alcohols such as 2-ethylhexanol, 2-propylheptanol, isotridecanol, isononanol, C9-C17 oxoalcohols;
   alkoxylated linear C2- to C36-alcohols such as alkoxylated mixture of C6- to C22-fatty alcohols, preferably alkoxylated mixtures of C8- to C22-fatty alcohols, more preferably alkoxylated mixtures of C12- and C14-fatty alcohols, most preferably alkoxylated mixtures of C16- and C18-fatty alcohols;
   alkoxylated branched C3- to C36-alcohols such as alkoxylated 2-ethylhexanol, alkoxylated 2-propylheptanol, alkoxylated isotridecanol, alkoxylated isononanol, alkoxylated C9-C17 oxoalcohols;
(b) di-alcohols such as alkane diols, polyalkoxylated C2-C6-alkanediols bearing at least two hydroxy groups,
(c) oligo-alcohols such as sugar alcohols, polyalkoxylated sugar alcohols, C3-C6-alkanetriols, polyalkoxylated C3-C6-alkanetriols, bearing at least three hydroxy groups,
(d) polyols such as C5-C6-alkane polyols, polyalkoxylated C5-C6-alkane polyols, polyetherols such as polyglycerol or di- or tri-pentaerythritol, alkoxylated polyetherols such as alkoxylated polyglycerol, alkoxylated di- or tri-pentaerythritol,
(e) phenoxyalkanols such as phenoxyethanol;
with the alcohol(s) selected from the groups of mono-alcohols and alkoxylated di-, oligo-alcohols and alkoxylated polyols being preferred, and the alcohols selected from the group(s) mono-alcohols and alkoxylated di-alcohols being even more preferred.

In one embodiment of the present disclosure, at least one linear or branched C₂- to C₃₆-alcohol containing at least one hydroxy group is used. In a preferred embodiment thereof at least one C₈- to C₂₂-fatty alcohol containing at least one hydroxy group is used. In a more preferred embodiment thereof a mixture of C₁₆- and C₁₈-fatty alcohols each containing at least one hydroxy group is used. In another more preferred embodiment thereof a mixture of C₁₈- and C₂₂-fatty alcohols each containing at least one hydroxy group is used. In another more preferred embodiment thereof at least one branched C₉- to C₁₇ alcohol is used. In a further, more preferred embodiment thereof linear or branched C₈- to C₁₀-mono-alcohols containing at least one hydroxy group are used. In a further even more preferred embodiment thereof 2-propylheptanol or 2-ethylhexanol are used. In another even more preferred embodiment thereof 2-ethylhexanol is used. In a further preferred embodiment of the present disclosure, at least one phenoxyalkanol is used. In another more preferred embodiment phenoxyethanol is used.

Obviously, such alcohols are known, many of them from natural sources. Others can be produced starting from natural sources by modifying them. If such modification does result in the addition of carbon-atoms, e.g., by chemical addition reactions, and if such addition reactions use non-fossil carbon atoms, then alcohols contain only non-fossil-based carbon. Hence, it is preferred, that only naturally occurring alcohol and/or nature-derived alcohols (not being made with the addition of non-fossil carbon in building blocks to obtain such nature-derive alcohols) are employed in the process detailed herein. Even more preferably, only naturally occurring alcohols are employed.

In a more preferred embodiment, the alcohol (A) employed for the esteramine, and the inventive process is an alkoxylated alcohol. Such alkoxylated alcohol is typically and thus preferably obtained by alkoxylating at least one hydroxy group of the alcohol as defined herein before with one or more alkylene oxides to produce alkylene oxy-chains comprising one or more moieties stemming from alkylene oxides selected from C2 to C22-alkylene oxides, preferably C2-C4-alkylene oxides, whereas the moieties stemming from the alkylene oxide(s) may be arranged in random, block or multiblock-order or combinations thereof, preferably as block.

In one embodiment of the present disclosure, alkyl alcohols alkoxylated with only a single alkylene oxide are used. In a further embodiment, alkyl alcohols alkoxylated with a first alkylene oxide followed by alkoxylation with a second alkylene oxide, thereby forming a block structure of different alkylene oxide blocks, are used.

Within the context of the details herein, it is also preferred that in case compound (A) comprises an alkoxylated alcohol comprises the alkoxylated fragment being based on at least one C2-C22 alkylene oxide, preferably C2-C4-alkylene oxides, more preferably ethylene oxide and/or propylene oxide, most preferably the respective alcohol comprises at least one block based on ethylene oxide and/or propylene oxide, and even more preferably contains only one block consisting of ethylene oxide or consisting of two blocks with the first block—preferably the “inner block” directly linked to the hydroxy group of the alcohol—consisting of ethylene oxide and a second block—preferably being the “outer block linked to the ethylene oxide-block—consisting of propylene oxide. More preferably, the EO-block comprises 3 to 10 EO-derived moieties and the PO-block comprises 1 to 10 PO-derived moieties. Most preferably, only ethylene oxide is employed.

The alkoxylation of the alcohol can be achieved by either carrying out the alkoxylation reaction with only one alkylene oxide or with more than one alkylene oxide. If more than one alkylene oxide is used, the resulting alkylether alcohols comprises either randomly distributed alkylene oxide units or a block of one alkylene oxide followed by a block of another alkylene oxide or a block of one alkylene oxide followed by another block which comprises two or more alkylene oxides arranged in random order or a block comprising two or more alkylene oxides is followed by another block which comprises two or more alkylene oxides with each such block being different in their relative amount of alkylene oxides, their arrangement of alkylene oxides and/or the identity of the alkylene oxides such that the two blocks linked to each other differ in their chemical composition and/or arrangement; any such combination of arrangements is in principle possible, and as such is encompassed by this present disclosure.

In one embodiment disclosed herein, alkyl alcohols alkoxylated with only a single alkylene oxide are used. In a further embodiment, alkyl alcohols alkoxylated with a first alkylene oxide followed by alkoxylation with a second alkylene oxide, thereby forming a block structure of different alkylene oxide blocks, are used.

It is clear that when switching from one block to the next there might be—depending on how the alkoxylation reaction is performed—there might be a relatively small “region” in between the blocks which do not have a sharp “border” between the block which is identical on every molecule of the alkoxylated monoalcohol, such that there might be some “dirty” structures which means that some very small amounts (such as a single or perhaps two or three) alkylene oxides of the first type employed might be inserted only after one or more of the second alkylene oxide have reacted; this depends mainly on the conversion rate achieved for the first alkylation reaction at the time point when the second alkylene oxide is added, i.e. if at that time of adding the second alkylene oxide still some unreacted amounts of the first alkylene oxide are present.

Similarly, it is clear that due to the statistical reaction of the alkoxylation polymerization the block lengths might differ slightly from individual compound to the next, and thus the “the alkoxylated monoalcohol,” in fact is a mixture of compounds having slightly differing chain lengths and block lengths.

This alkoxylation reaction may be undertaken generally in the presence of a catalyst at a reaction temperature from about 70 to about 200° C., in another embodiment from about 80 to about 160° C. This reaction may be affected at a pressure of up to about 10 bar, in another embodiment at a pressure of up to about 8 bar.

Examples of suitable catalysts comprise basic catalysts such as alkali metal and alkaline earth metal hydroxides such as sodium hydroxide, potassium hydroxide and calcium hydroxide, alkali metal alkoxides, in particular sodium and potassium C₁-C₄-alkoxides, such as sodium methoxide, sodium ethoxide and potassium tert-butoxide, alkali metal and alkaline earth metal hydrides such as sodium hydride and calcium hydride, and alkali metal carbonates such as sodium carbonate and potassium carbonate. In one embodiment, alkali metal hydroxides are used. In another embodiment, potassium hydroxide and sodium hydroxide are used. Typical use amounts for the base are from 0.01 to 10% by weight, or from 0.05 to 2% by weight, based on the total amount of alcohol and C₂- to C₂₂ alkylene oxide, preferably C2-C4-alkylene oxides.

It is noted that the alkylene oxide used to prepare the alkoxylated alcohol(s) may be derived from a fossil or non-fossil carbon source or even a mixture thereof. Preferably, the amount of non-fossil carbon atoms in the alkylene oxide employed is at least 10%, at least 20%, at least 40%, at least 70%, at least 95% and most preferably up top 100% based on non-fossil derived carbon atoms; the same applies to the total inventive compound as such. The skilled person is well-aware of commercial alkylene oxide products made of non-fossil carbon sources (these products are often sold as being “sustainable”, “renewable” or “bio-based”). For example, Croda International, Snaith, UK, sells ethylene oxide and related products based on bio-ethanol as “ECO”-Range. Additionally, methods to prepare bio-based propylene oxide are also known (see Abraham, D. S., “Production of propylene oxide from propylene glycol” Master's Thesis University of Missouri-Columbia (2007) (75 pages)).

The acid (C) for the inventive esteramine and their salts and the inventive process is selected from:
i) sulfonic acids, such as alkyl sulfonic acids such as methanesulfonic acid, ethylsulfonic acid, propylsulfonic acid, camphorsulfonic acid; alkylarylsulfonic acids and specifically alkylbenzenesulfonic acids, such as toluenesulfonic acid (including the mixture of isomers thereof), p-toluenesulfonic acid, o-toluenesulfonic acid, m-toluenesulfonic acid, xylenesulfonic acid (mixture of isomers), 2, 6-dimethylbenzenesulfonic acid, 2,5-dimethylbenzenesulfonic acid, 2, 4-dimethylbenzenesulfonic acid, 4-dodecylbenzenesulfonic acid, iso-propyl benzenesulfonic acid, ethylbenzenesulfonic acid, and naphthalenesulfonic acid; and
ii) inorganic acids such as hydrochloric acid, hydrobromic acid, phosphoric acid.

The acids employed preferably are concentrated such that the amount of water introduced into the reaction is minimized or are employed as solids. As such, also gases such as hydrogen chloride may be employed as this can be introduced as gas. In one embodiment for the inventive esteramine and the inventive process, the acid is preferably selected from group i), more preferably p-toluenesulfonic acid and/or methanesulfonic acid, most preferably methanesulfonic acid.

In case the amount of water introduced into the reaction via the acid, water can be removed using known means such as distillative procedures and equipment (such as a Dean-Stark-trap and the like) before the orthoester is introduced.

The esteramine is obtained as a at least partially protonated salt in cationic form.

In a preferred embodiment, methanesulfonic acid is used as concentrated methanesulfonic acid. In another preferred embodiment, methanesulfonic acid is used as about 70 wt.-% methanesulfonic acid solution in water or as “pure” acid, e.g., typically close to 100 wt. % purity (equal to “about 100 wt. %”); any concentration in between however can also be employed. In a further preferred embodiment methanesulfonic acid is used as about 70 wt.-% methanesulfonic acid-solution in water. However, in the most preferred embodiment, the sulfonic acid such as the methanesulfonic acid is introduced as solid, even more preferably as dried solid (to reduce the amount of water introduced into the reaction).

In one embodiment of the present disclosure, the total amount of acid (C) is added at the beginning of the reaction. In another embodiment the acid is added dropwise for a certain period during the reaction, provided that during the reaction the acid (C) is always present.

The present disclosure can also provide inventive esteramines and their salts starting from natural amino acids and/or natural alcohols, and thus enables providing products with high content of bio-derived material. As the alkylene oxide(s) can be also derived from non-fossil sources, the inventive compound can be obtained containing only carbon-atoms from non-fossil sources. Such products are being highly sought after by consumers and industries.

### Treatment Compositions:

The treatment compositions detailed herein include at least one perfume raw material and an esteramine or salts thereof, the esteramine or salt thereof preferably produced as described above. Treatment compositions that employ the esteramines and salts thereof that are obtained by the reaction processes detailed herein will have less residual fatty alcohols (i.e., alcohol (A) as defined herein), when compared with esteramines and salts thereof that are obtained by different, reaction processes. Accordingly, due to having less residual fatty alcohols in the treatment composition, the treatment composition will have a better base odor. This can be confirmed through GC/MS (gas chromatography/mass spectrometry) headspace analysis comparing treatment compositions that only vary in the source of esteramines and salts thereof employed, and the employed esteramines and salts thereof will vary by the process in which they are derived.

The treatment compositions may be consumer product compositions. The consumer products compositions of the present disclosure may be useful in baby care, beauty care, fabric care, home care, family care, feminine care, and/or health care applications. The treatment compositions may be useful for treating a surface, such as fabric, hair, or skin. The consumer product compositions may be intended to be used or consumed in the form in which it is sold. The consumer product compositions may be not intended for subsequent commercial manufacture or modification.

The treatment composition may be a household care composition. The treatment composition may be a fabric care composition, a hard surface cleaner composition, a dish care composition, a hair care composition (such as shampoo or conditioner), a body cleansing composition, or a mixture thereof.

The treatment composition may be a fabric care composition, such as a laundry detergent composition (including a heavy-duty liquid washing detergent or a unit dose article), a fabric conditioning composition (including a liquid fabric softening and/or enhancing composition), a laundry additive, a fabric pre-treat composition (including a spray, a pourable liquid, or a spray), a fabric refresher composition (including a spray), or a mixture thereof.

The treatment composition may be a beauty care composition, such as a hair treatment product (including shampoo and/or conditioner), a skin care product (including a cream, lotion, or other topically applied product for consumer use), a shave care product (including a shaving lotion, foam, or pre- or post-shave treatment), personal cleansing product (including a liquid body wash, a liquid hand soap, and/or a bar soap), a deodorant and/or antiperspirant, or mixtures thereof.

The treatment composition may be a home care composition, such as an air care, car care, dishwashing, hard surface cleaning and/or treatment, and other cleaning for consumer or institutional use.

The treatment composition may be in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof.

The treatment composition may be in the form of a liquid. The liquid composition may include from about 30%, or from about 40%, or from about 50%, to about 99%, or to about 95%, or to about 90%, or to about 75%, or to about 70%, or to about 60%, by weight of the composition, of water. The liquid composition may be a liquid laundry detergent, a liquid fabric conditioner, a liquid dish detergent, a hair shampoo, a hair conditioner, or a mixture thereof.

The treatment composition may be in the form of a solid. The solid composition may be a powdered or granular composition. Such compositions may be agglomerated or spray-dried. Such composition may include a plurality of granules or particles, at least some of which include comprise different compositions. The composition may be a powdered or granular cleaning composition, which may include a bleaching agent. The composition may be in the form of a bead or pastille, which may be made into a bead or pastille from a liquid melt. The composition may be an extruded product.

The treatment composition may be in a particulate form, such as a plurality of particulates. Individual particulates may have a mass from about 1 mg to about 1 g. The emulsion may be dispersed in a water-soluble carrier. The water-soluble carrier may be selected from the group consisting of polyethylene glycol, sodium acetate, sodium bicarbonate, sodium chloride, sodium silicate, polypropylene glycol polyoxoalkylene, polyethylene glycol fatty acid ester, polyethylene glycol ether, sodium sulfate, starch, and mixtures thereof. The water-soluble carrier may be a water-soluble polymer. The treatment composition, when in particulate form, may comprise from about 25 wt % to about 99.99 wt % of the water-soluble carrier, and from about 0.001 wt % to about 50 wt % by weight the esteramine or salt thereof. The particulate form may be in the form of a bead or pastille.

The treatment composition may be in the form of a unitized dose article, such as a tablet, a pouch, a sheet, or a fibrous article. Such pouches typically include a water-soluble film, such as a polyvinyl alcohol water-soluble film, that at least partially encapsulates a composition. Suitable films are available from MonoSol, LLC (Indiana, USA). The composition can be encapsulated in a single or multi-compartment pouch. A multi-compartment pouch may have at least two, at least three, or at least four compartments. A multi-compartmented pouch may include compartments that are side-by-side and/or superposed. The composition contained in the pouch or compartments thereof may be liquid, solid (such as powders), or combinations thereof. Pouched compositions may have relatively low amounts of water, for example less than about 20%, or less than about 15%, or less than about 12%, or less than about 10%, or less than about 8%, by weight of the detergent composition, of water.

The treatment composition may be in the form of a spray and may be dispensed, for example, from a bottle via a trigger sprayer and/or an aerosol container with a valve.

The treatment composition may have a viscosity of from 1 to 1500 centipoises (1-1500 mPa*s), from 100 to 1000 centipoises (100-1000 mPa*s), or from 200 to 500 centipoises (200-500 mPa*s) at 20 s⁻¹ and 21° C.

The treatment composition may include from about 0.001% to about 30%, preferably from about 0.001% to about 20%, more preferably from about 0.001% to about 15%, 0.001% to about 10%, more preferably from about 0.01% to about 5%, more preferably from about 0.1% to about 3%, more preferably from about 0.5% to about 2%, by weight of the treatment composition, of the esteramine or salt thereof that has been produced by the processes as described herein.

The treatment composition according may comprise a ratio of orthoester to esteramine or salt thereof of about 1:20 to about 1:1, more preferably about 1:15 to about 1:5, more preferably about 1:12 to about 1:8, more preferably about 1:10.

The treatment composition may include an orthoester at a level of from about 0.0001% to about 3%, by weight of the treatment composition, more preferably about 0.001% to about 0.5%, more preferably about 0.01% to about 0.3%, even more preferably about 0.05% to about 0.2%.

The treatment composition may include from about 0.001% to about 30%, preferably from about 0.001% to about 20%, more preferably from about 0.001% to about 15%, 0.001% to about 10%, more preferably from about 0.01% to about 5%, more preferably from about 0.1% to about 3%, more preferably from about 0.5% to about 2%, by weight of the treatment composition, of at least one perfume raw material.

The at least one perfume raw material may include an aldehyde moiety, a ketone moiety, or a mixture thereof. The term “perfume raw material” (or “PRM”) as used herein refers to compounds that may have a molecular weight of at least about 100 g/mol (optionally up to about 1000 g/mol, preferably up to about 500 g/mol) and which are useful in imparting an odor, fragrance, essence, or scent, either alone or with other perfume raw materials. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

Perfume raw materials that comprise an aldehyde moiety are provided below in Table B. It is believed that the materials provided in Table B are illustrative (but non-limiting) examples of PRMs that are suitable for use according to the present disclosure.

TABLE B
Aldehyde-containing perfume raw materials.

| Number | Registry Name | Trade Name |
|---|---|---|
| 1 | 3-Cyclohexene-1-carboxaldehyde, dimethyl- | Ligustral |
| 2 | 3-Cyclohexene-1-carboxaldehyde, 2,4,6- | Isocyclocitral |
| | trimethyl- | |
| 3 | Cyclohexanemethanol, .alpha.,3,3-trimethyl-, | Aphermate |
| | formate | |
| 4 | 3-(4-tert-butylphenyl)butanal; pt-bucinal; 3-(4- | Lilial |
| | tert-butylphenyl)butanal | |
| 5 | 2-methylundecanal | Methyl Nonyl Acetaldehyde |
| 6 | 1-methyl-3-(4-methylpent-3-enyl)cyclohex-3- | Precyclemone B |
| | ene-1-carbaldehyde; myrmac aldehyde | |
| 7 | Benzenepropanal, 3-(4-ethylphenyl)-2,2- | Floralozone |
| | dimethylpropanal | |
| 8 | 2,4-dimethylcyclohex-3-ene-1-carbaldehyde | Ligustral/Triplal |
| 9 | Decanal | Decyl Aldehyde |
| 10 | 10-Undecen-1-al; Undecenoic aldehyde; n- | Undecylenic aldehyde; |
| | Undecenoic aldehyde; Hendecen-10-al; | Aldehyde C-11, |
| | | unsaturated; Aldehyde C-11 |
| | | undecylenic; |
| 11 | 8-,9 and 10-Undecenal, mixture of isomers | Intreleven aldehyde |
| 12 | Benzenepropanal, .alpha.-methyl-4-(1- | Cylamen Aldehyde; |
| | methylethyl)- | |
| 13 | 2,6,10-trimethylundec-9-enal | Adoxal; Farenal |
| 14 | 4-(octahydro-4,7-methano-5H-inden-5- | Dupical |
| | ylidene)butanal | |
| 15 | 3-Ethoxy-4-hydroxybenzaldehyde | Ethyl vanillin |
| 16 | tricyclo[5.2.1.02, 6]decane-3-carbaldehyde | Vertral ® |
| 17 | 4,7-Methano-1H-indene-2-carboxaldehyde, | Scentenal ® 981810 |
| | octahydro-5-methoxy-; 6-Methoxy | |
| | dicyclopentadiene carboxaldehyde; 8- | |
| | Methoxytricyclo(5.2.2.1)decane-4- | |
| | carboxaldehyde; Octahydro-5-methoxy-4,7- | |
| | methano-1H-indene-2-carboxaldehyde; | |
| 18 | 4-Hydroxy-3-methoxybenzaldehyde | Vanillin |
| 19 | Trans-4-decenal | Decenal-4-trans |
| 20 | α-hexyl-; α-n-Hexyl-β-phenylacrolein; 2- | α-Hexylcinnamaldehyde; α- |
| | Hexyl-3-phenyl-2-propenal; 2-Hexyl-3-phenyl- | Hexylcinnamic aldehyde; Hexyl |
| | propenal; (2Z)-2-Hexyl-3-phenyl-2-propenal; | cinnamic aldehyde; |
| | Hexyl-3-phenyl-propenal; n-Hexyl | Hexylcinnamaldehyde; |
| | cinnamaldehyde; (2E)-2-Benzylideneoctanal; 2- | Cinnamaldehyde, |
| | [(E)-Benzylidene]octanal | |
| 21 | 4-Dodecenal | Tangerinal DIPG 984655 |
| 22 | 3-Cyclohexene-1-propanal,beta,4-dimethyl- | Liminal ® 955374 |
| 23 | trans-2-Dodecenal | Mandarine aldehyde 10% CITR |
| | | 965765 |
| 24 | 4,8-Dimethyl-4,9-decadienal | Floral Super |
| 25 | Hydroxymyrac aldehyde; 4-(4-Hydroxy-4- | Lyral |
| | methyl-pentyl)-3-cyclohexen-1- | |
| | carboxyaldehyde; Lyral; Kovanol | |
| 26 | 2-Hexenal, (E)- | 2-Hexenal |
| 27 | Benzaldehyde | Benzaldehyde |
| 28 | Benzeneacetaldehyde | Phenyl Acetaldehyde |
| 29 | Benzeneacetaldehyde, .alpha.-methyl- | Hydratropic Aldehyde |
| 30 | 3-Cyclohexene-1-carboxaldehyde, 3,5- | Cyclal C, |
| | dimethyl- | |
| 31 | Benzaldehyde, 4-methoxy- | Anisic Aldehyde |
| 32 | Octanal, 7-hydroxy-3,7-dimethyl- | Hydroxycitronellal |
| 33 | 3-Cyclohexene-1-carboxaldehyde, 3,6- | Cyclovertal |
| | dimethyl- | |
| 34 | Octanal, 7-methoxy-3,7-dimethyl- | Methoxycitronellal Pq |
| 35 | Benzenepropanal, beta.-methyl-; 3- | Trifernal |
| | phenylbutanal | |
| 36 | 4,7-Methano-1H-indenecarboxaldehyde, | Formyltricyclodecan |
| | octahydro- | |
| 37 | Octanal | Octyl Aldehyde |
| 38 | 5-Heptenal, 2,6-dimethyl- | Melonal |
| 39 | Octanal, 3,7-dimethyl- | Dihydrocitronellal |
| 40 | 2-Nonenal | 2 Nonen-1-al |
| 41 | 6-Octenal, 3,7-dimethyl- | Citronellal |
| 42 | 2-Decenal | 2 Decene-1-al |
| 43 | 2,6-Octadienal, 3,7-dimethyl- | Citral |
| 44 | Undecenal | Iso C-11 Aldehyde |
| 45 | Undecanal | Undecyl Aldehyde |
| 46 | 2-Undecenal | 2-Undecene-1-Al |
| 47 | Benzaldehyde, 4-(1-methylethyl)- | Cuminic Aldehyde |
| 48 | Decanal, 2-methyl- | Methyl Octyl Acetaldehyde |
| 49 | Benzenepropanal, 4-(1,1-dimethylethyl)- | Bourgeonal |
| 50 | 2-Dodecenal | 2 Dodecene-1-al |
| 51 | Benzenepropanal, .beta.-methyl-3-(1- | Florhydral |
| | methylethyl)- | |
| 52 | 1,3-Benzodioxole-5-carboxaldehyde | Heliotropin |
| 53 | 3-Cyclohexene-1-carboxaldehyde, 1-methyl-4- | Vernaldehyde |
| | (4-methylpentyl)- | |
| 54 | Benzenepropanal, 4-methoxy-.alpha.-methyl- | Canthoxal |
| 55 | Cyclohexenebutanal, .alpha.,2,2,6-tetramethyl- | Cetonal |
| 56 | Dodecanal | Lauric Aldehyde |
| 57 | 5,9-Undecadienal, 2,6,10-trimethyl- | Oncidal |
| 58 | Bicyclo[2.2.2]oct-5-ene-2-carboxaldehyde, 6- | Maceal |
| | methyl-8-(1-methylethyl)- | |
| 59 | 2-methyl-3-[4-(2- | cyclamen homoaldehyde |
| | methylpropyl)phenyl]propanal | |
| 60 | 6-methoxy-2,6-dimethyloctanal | calypsone |
| 61 | 4-propan-2-ylbenzaldehyde | Cuminic Aldehyde |
| 62 | 3,6-dimethylcyclohex-3-ene-1-carbaldehyde | VERTOLIFF |
| 63 | 2-methyl-3-(4-methylphenyl)propanal | Jasmorange ®; satinaldehyde |
| 64 | 3-phenylprop-2-enal | Cinnamic Aldehyde |

The perfume raw materials that may be utilized in the treatment compositions detailed herein may be selected from the group consisting of the aldehyde-containing PRMs of Table A, above. The PRMs useful herein may comprise an aldehyde moiety and preferably be selected from the group consisting of: methyl nonyl acetaldehyde: benzaldehyde; floralozone; isocyclocitral; triplal (ligustral); precylcemone B; lilial; decyl aldehyde; undecylenic aldehyde; cyclamen homoaldehyde; cyclamen aldehyde; dupical; oncidal; adoxal; melonal; calypsone; anisic aldehyde; heliotropin; cuminic aldehyde; scentenal; 3,6-dimethylcyclohex-3-ene-1-carbaldehyde; satinaldehyde; canthoxal; vanillin; ethyl vanillin; cinnamic aldehyde; cis-4-decenal; trans-4-decenal; cis-7-decenal; undecylenic aldehyde; trans-2-hexenal; trans-2-octenal; 2-undecenal; 2,4-dodecadeienal; cis-4-heptenal; Florydral; butyl cinnamaldehyde; limonelal; amyl cinnamaldehyde; hexyl cinnamaldehyde; citronellal; citral; cis-3-hexen-1-al; and mixtures thereof.

As mentioned above, the perfume raw materials that may be useful in the treatment compositions detailed herein may comprise a ketone moiety. Perfume raw materials that comprise a ketone moiety are provided below in Table C. It is believed that the materials provided in Table C are illustrative (but non-limiting) examples of PRMs that are suitable for use according to the present disclosure.

TABLE C
Ketone-containing perfume raw materials.

| Number | Registry Name | Trade Name |
|---|---|---|
| 1 | 2-Buten-1-one, 1-(2,6,6-trimethyl-3- | delta-Damascone |
| | cyclohexen-1-yl)- | |
| 2 | (1-(2,6,6-Trimethyl-2-cyclohexen-1-yl)-2- | alpha-Damascone |
| | buten-1-one); 2-Buten-1-one, 1-(2,6,6- | |
| | trimethyl-2-cyclohexen-1-yl)-, (E)- | |
| 3 | (1-(2,6,6-Trimethyl-1-cyclohexen-1-yl)-2- | beta-Damascone |
| | buten-1-one); 2-Buten-1-one, 1-(2,6,6- | |
| | trimethyl-1-cyclohexen-1-yl)-, (E)- | |
| 4 | 2-Buten-1-one, 1-(2,6,6-trimethyl-1,3- | Damascenone |
| | cyclohexadien-1-yl)- | |
| 5 | 1,1,2,3,3-pentamethyl-2,5,6,7-tetrahydroinden- | Cashmeran |
| | 4-one | |
| 6 | 1-(5,5-dimethyl-1-cyclohexenyl)pent-4-en-1- | Neobutenone Alpha |
| | one | |
| 7 | 1-(5,5-dimethyl-1-cyclohexenyl)pent-4-en-1- | Galbascone; Dynascone |
| | one | |
| 8 | 1-naphthalen-2-ylethanone | Methyl Beta-Naphthyl Ketone |
| 9 | 2-(2-(4-Methyl-3-cyclohexen-1- | Nectaryl |
| | yl)propyl)cyclo-pentanone | |
| 10 | 2-Hexyl-2-cyclopenten-1-one (main | Isojasmone B 11 |
| | component) | |
| 11 | Methyl 2,6,10-Trimethyl-2,5,9- | Trimofix “O” |
| | cyclododecatrien-1-yl ketone; | |
| 12 | α-Isomethyl ionone; 5-(2,6,6-Trimethyl-2- | Methyl ionone; Methyl Ionone |
| | cyclohexen-1-yl)-3-methyl-3-buten-2-one; | Alpha Iso; Methyl Ionone |
| | | Gamma; Isoraldeine 70; |
| | | Isoraldeine 95; Gamma |
| | | Methylionone 600 UC; Alpha |
| | | Daphnone; Iraldeine gamma; |
| | | gamma Methyl Ionone Pure; |
| | | gamma Methyl Ionone A; |
| | | Gamma Methyl Ionone Coeur |
| 13 | 2-Heptylcyclopentanone; | Fleuramone; Projasmon |
| 14 | 3-(Hydroxymethyl)nonan-2-one (and isomer) | Methyl lavender ketone |
| 15 | 2-Cyclohexen-1-one, 2-methyl-5-(1- | Laevo Carvone |
| | methylethenyl)-, (R)- | |
| 16 | Bicyclo[2.2.1]heptan-2-one, 1,7,7-trimethyl-, | Camphor Gum |
| | (1R)- | |
| 17 | 2-Heptanone | Methyl Amyl Ketone |
| 18 | 3-Octanone | Ethyl Amyl Ketone |
| 19 | 2-Octanone | Methyl Hexyl Ketone |
| 20 | 5-Hepten-2-one, 6-methyl- | Methyl Heptenone |
| 21 | Ethanone, 1-(4-methylphenyl)- | Para Methyl Acetophenone |
| 22 | 2-Butanone, 4-phenyl- | Benzyl Acetone |
| 23 | 1,4-Methanonaphthalen-5(1H)-one, | Tamisone |
| | 4,4a,6,7,8,8a-hexahydro- | |
| 24 | 2H-1-Benzopyran-2-one, 3,4-dihydro- | Dihydrocoumarin |
| 25 | Cyclohexanone, 5-methyl-2-(1-methylethyl)-, | Iso Menthone |
| | cis- | |
| 26 | 2H-Pyran-2-one, 6-butyltetrahydro- | Nonalactone |
| 27 | 3-Hepten-2-one, 3,4,5,6,6-pentamethyl- | Koavone |
| 28 | Cyclopentanone, 3-methyl-2-pentyl- | Jasmylone |
| 29 | 3-Nonanone | Ethyl Hexyl Ketone |
| 30 | Ethanone, 1-(3,3-dimethylcyclohexyl)- | Herbac |
| 31 | 3-Heptanone, 5-methyl-, oxime | Stemone |
| 32 | Cyclohexanone, 2-(1-methylpropyl)- | 2-Sec-Butyl Cyclo Hexanone |
| 33 | Cyclopentanone, 2-pentyl- | Delphone |
| 34 | 2-Cyclopenten-1-one, 3-methyl-2-pentyl- | Dihydrojasmone |
| 35 | Cyclohexanone, 5-methyl-2-(1-methylethyl)-, | Menthone Racemic |
| | trans- | |
| 36 | Cyclohexanone, 4-(1,1-dimethylpropyl)- | Orivone |
| 37 | 2-Undecanone | Methyl Nonyl Ketone |
| 38 | 1-Decanol | Rhodalione |
| 39 | 2-Cyclohexen-1-one, 3-methyl-5-propyl- | Livescone |
| 40 | 2-Cyclopenten-1-one, 2-methyl-3-(2-pentenyl)- | Iso Jasmone |
| 41 | Ionone | Ionone Ab |
| 42 | 3-Buten-2-one, 4-(2,6,6-trimethyl-2- | Ionone Alpha |
| | cyclohexen-1-yl)-, (E)- | |
| 43 | 3-Buten-2-one, 4-(2,6,6-trimethyl-1- | Ionone Beta |
| | cyclohexen-1-yl)- | |
| 44 | 2-Buten-1-one, 1-(2,4,4-trimethyl-2- | Isodamascone N |
| | cyclohexen-1-yl)-, (E)- | |
| 45 | 2H-1-Benzopyran-2-one | Coumarin |
| 46 | Cyclopentanone, 2-heptyl- | Fleuramone |
| 47 | 3-Decanone, 1-hydroxy- | Methyl Lavender Ketone |
| 48 | 1-Propanone, 1-[2-methyl-5-(1-methylethyl)-2- | Nerone |
| | cyclohexen-1-yl]- | |
| 49 | 9-Undecen-2-one, 6,10-dimethyl- | Tetra Hydro Psuedo Ionone |
| 50 | 1-phenylethanone | Acetophenone |
| 51 | 2-butan-2-ylcyclohexan-1-one | Freskomenthe |
| 52 | Ethanone, 1-(3-methyl-2-benzofuranyl)- | nerolione |
| 53 | 4-(4-methoxyphenyl)butan-2-one | Anisyl Acetone |

The perfume raw materials that may be utilized in the treatment compositions detailed herein may be selected from the group consisting of the ketone-containing PRMs of Table C, above. The PRMs useful herein may comprise a ketone moiety and may preferably be selected from the group consisting of: nerolione; 4-(4-methoxyphenyl) butan-2-one; 1-naphthalen-2-ylethanone; nectaryl; trimofix O; fleuramone; delta-damascone; beta-damascone; alpha-damascone; methyl ionone; 2-hexylcyclopent-2-en-1-one; galbascone; and mixtures thereof.

The treatment compositions detailed herein may utilize a perfume raw material selected from the group consisting of: methyl nonyl acetaldehyde: benzaldehyde; floralozone; isocyclocitral; triplal (ligustral); precylcemone B; lilial; decyl aldehyde; undecylenic aldehyde; cyclamen homoaldehyde; cyclamen aldehyde; dupical; oncidal; adoxal; melonal; calypsone; anisic aldehyde; heliotropin; cuminic aldehyde; scentenal; 3,6-dimethylcyclohex-3-ene-1-carbaldehyde; satinaldehyde; canthoxal; vanillin; ethyl vanillin; cinnamic aldehyde; cis-4-decenal; trans-4-decenal; cis-7-decenal; undecylenic aldehyde; trans-2-hexenal; trans-2-octenal; 2-undecenal; 2,4-dodecadeienal; cis-4-heptenal; Florydral; butyl cinnamaldehyde; limonelal; amyl cinnamaldehyde; hexyl cinnamaldehyde; citronellal; citral; cis-3-hexen-1-al; nerolione; 4-(4-methoxyphenyl) butan-2-one; 1-naphthalen-2-ylethanone; nectaryl; trimofix O; fleuramone; delta-damascone; beta-damascone; alpha-damascone; methyl ionone; 2-hexylcyclopent-2-en-1-one; galbascone; and mixtures thereof.

In the treatment compositions detailed herein, for the perfume raw materials utilized, it may be preferred that the hydrophobic moiety is an organic group comprising from about 8 to about 18 chain atoms, more preferably from about 10 to about 18 chain atoms, preferably wherein the chain atoms are carbon atoms. It is believed that such chain lengths provide a suitable degree of hydrophobicity to facilitate deposition benefits, particularly in laundry applications where the treatment compositions described herein are aqueous liquors.

The perfume raw materials in this specification, including the perfume raw materials listed above, can be obtained from various suppliers including: International Flavors and Fragrances of New York, NY USA; Givaudan of Vernier Switzerland; Firmenich of Geneva, Switzerland; Symrise of Holzminden, Germany; Kao of Tokyo, Japan; Takasago of Tokyo, Japan; and Florasynth of Tel-Aviv, Israel.

### Adjunct Ingredients

The treatment compositions of the present disclosure, which may be consumer products, may comprise an adjunct material. The adjunct material may provide a benefit in the intended end-use of a composition, or it may be a processing and/or stability aid.

Suitable adjunct materials may include: surfactants, conditioning actives, deposition aids, rheology modifiers or structurants, antioxidants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, perfume delivery systems (such as core/shell encapsulates, other pro-fragrance materials, and the like), structure elasticizing agents, carriers, hydrotropes, processing aids, anti-agglomeration agents, coatings, formaldehyde scavengers, and/or pigments.

Depending on the intended form, formulation, and/or end-use, compositions of the present disclosure or may not may not contain one or more of the following adjunct materials: surfactants, conditioning actives, deposition aids, rheology modifiers or structurants, antioxidants, bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids, structurants, anti-agglomeration agents, coatings, formaldehyde scavengers and/or pigments.

The precise nature of these additional components, and levels of incorporation thereof, will depend on the physical form of the composition and the nature of the operation for which it is to be used. However, when one or more adjuncts are present, such one or more adjuncts may be present as detailed below. The following is a non-limiting list of suitable additional adjuncts.

### A. Surfactants

The treatment compositions of the present disclosure may comprise surfactant. Surfactants may be useful for providing, for example, cleaning benefits. The compositions may comprise a surfactant system, which may contain one or more surfactants.

The treatment compositions of the present disclosure may include from about 0.1% to about 70%, or from about 2% to about 60%, or from about 5% to about 50%, by weight of the composition, of a surfactant system. Liquid compositions may include from about 5% to about 40%, by weight of the composition, of a surfactant system. Compact formulations, including compact liquids, gels, and/or compositions suitable for a unit dose form, may include from about 25% to about 70%, or from about 30% to about 50%, by weight of the composition, of a surfactant system.

The surfactant system may include anionic surfactant, nonionic surfactant, zwitterionic surfactant, cationic surfactant, amphoteric surfactant, or combinations thereof. The surfactant system may include linear alkyl benzene sulfonate, alkyl ethoxylated sulfate, alkyl sulfate, nonionic surfactant such as ethoxylated alcohol, amine oxide, or mixtures thereof. The surfactants may be, at least in part, derived from natural sources, such as natural feedstock alcohols.

Suitable anionic surfactants may include any conventional anionic surfactant. This may include a sulfate detersive surfactant, for e.g., alkoxylated and/or non-alkoxylated alkyl sulfate materials, and/or sulfonic detersive surfactants, e.g., alkyl benzene sulfonates. The anionic surfactants may be linear, branched, or combinations thereof. Preferred surfactants include linear alkyl benzene sulfonate (LAS), alkyl ethoxylated sulfate (AES), alkyl sulfates (AS), or mixtures thereof. Other suitable anionic surfactants include branched modified alkyl benzene sulfonates (MLAS), methyl ester sulfonates (MES), sodium lauryl sulfate (SLS), sodium lauryl ether sulfate (SLES), and/or alkyl ethoxylated carboxylates (AEC). The anionic surfactants may be present in acid form, salt form, or mixtures thereof. The anionic surfactants may be neutralized, in part or in whole, for example, by an alkali metal (e.g., sodium) or an amine (e.g., monoethanolamine).

The surfactant system may include nonionic surfactant. Suitable nonionic surfactants include alkoxylated fatty alcohols, such as ethoxylated fatty alcohols. Other suitable nonionic surfactants include alkoxylated alkyl phenols, alkyl phenol condensates, mid-chain branched alcohols, mid-chain branhed alkyl alkoxylates, alkylpolysaccharides (e.g., alkylpolyglycosides), polyhydroxy fatty acid amides, ether capped poly(oxyalkylated) alcohol surfactants, and mixtures thereof. The alkoxylate units may be ethyleneoxy units, propyleneoxy units, or mixtures thereof. The nonionic surfactants may be linear, branched (e.g., mid-chain branched), or a combination thereof. Specific nonionic surfactants may include alcohols having an average of from about 12 to about 16 carbons, and an average of from about 3 to about 9 ethoxy groups, such as C12-C14 EO7 nonionic surfactant.

Suitable zwitterionic surfactants may include any conventional zwitterionic surfactant, such as betaines, including alkyl dimethyl betaine and cocodimethyl amidopropyl betaine, C₈ to C₁₈ (for example from C₁₂ to C₁₈) amine oxides (e.g., C₁₂₋₁₄ dimethyl amine oxide), and/or sulfo and hydroxy betaines, such as N-alkyl-N,N-dimethylammino-1-propane sulfonate where the alkyl group can be C₈ to C₁₈, or from C₁₀ to C₁₄. The zwitterionic surfactant may include amine oxide.

Depending on the formulation and/or the intended end-use, the composition may be substantially free of certain surfactants. For example, liquid fabric enhancer compositions, such as fabric softeners, may be substantially free of anionic surfactant, as such surfactants may negatively interact with cationic ingredients.

### B. Conditioning Active

The treatment compositions of the present disclosure may include a conditioning active. Compositions that contain conditioning actives may provide softness, anti-wrinkle, anti-static, conditioning, anti-stretch, color, and/or appearance benefits.

Conditioning actives may be present at a level of from about 1% to about 99%, by weight of the composition. The composition may include from about 1%, or from about 2%, or from about 3%, to about 99%, or to about 75%, or to about 50%, or to about 40%, or to about 35%, or to about 30%, or to about 25%, or to about 20%, or to about 15%, or to about 10%, by weight of the composition, of conditioning active. The composition may include from about 5% to about 30%, by weight of the composition, of conditioning active.

Conditioning actives suitable for compositions of the present disclosure may include quaternary ammonium ester compounds, silicones, non-ester quaternary ammonium compounds, amines, fatty esters, sucrose esters, silicones, dispersible polyolefins, polysaccharides, fatty acids, softening or conditioning oils, polymer latexes, polyhydroxystearic acid and/or derivatives thereof, glyceride copolymers, or combinations thereof. Preferably, the conditioning active is a cationic conditioning active, which may improve the delivery/deposition of the esteramine or salt thereof.

The treatment composition may comprise a conditioning active, where the conditioning active comprises quaternary ammonium ester compounds. Preferably, the quaternary ammonium ester compounds are present at a level of from about 2 wt % to about 35 wt %, preferably from about 4 wt % to about 25 wt %, more 5 wt % to about 20 wt %, even more preferably from about 6 wt % to about 15 wt %, even more preferably from about 7 wt % to about 12 wt %, by weight of the treatment composition. The quaternary ammonium ester compounds (also known as “ester quats”) may be monoester quats, diester quats, triester quats, or a combination thereof; preferably, diester quat material forms the major portion (whether a majority or a plurality) of the ester quat compounds. It is believed that in addition to providing conditioning benefits, selecting the proper type and/or level of conditioning active (namely, a quaternary ammonium ester compound) can improve the deposition and/or performance of the esteramine or salt thereof described in the present disclosure.

The quaternary ammonium ester compound may comprise compounds according to the following formula:

$$\{ {R}_{( 4 - m )}^{2} - N + - {[ X - Y - {R}^{1} ]}_{m} \} {A}^{-}$$

wherein:
   m is 1, 2 or 3, with provisos that, in a given molecule, the value of each m is identical;
   each R¹, which may comprise from 13 to 22 carbon atoms, is independently a linear hydrocarbyl or branched hydrocarbyl group, preferably R¹ is linear, more preferably R¹ is partially unsaturated linear alkyl chain;
   each R² is independently a C₁-C₃ alkyl or hydroxyalkyl group and/or each R₂ is selected from methyl, ethyl, propyl, hydroxyethyl, 2-hydroxypropyl, 1-methyl-2-hydroxyethyl, poly(C₂-C₃ alkoxy), polyethoxy, benzyl, more preferably methyl or hydroxyethyl;
   each X is independently —(CH₂)n-, —CH₂—CH(CH₃)— or —CH(CH₃)—CH₂—, where each n is independently 1, 2, 3 or 4, preferably each n is 2;
   each Y is independently —O—(O)C— or —C(O)—O—; and
   A− is independently selected from the group consisting of chloride, bromide, methyl sulfate, ethyl sulfate, sulfate, and nitrate, preferably A− is selected from the group consisting of chloride and methyl sulfate, more preferably A− is methyl sulfate.

For monoester quats, m is 1. For diester quats, m is 2. For triester quats, m is 3. The conditioning active may comprise a mixture of monoester quats and diester quats, or even a mixture of monoester quats, diester quats, and triester quats. As will be appreciated by one of ordinary skill, the mixture may depend, in part, on the starting/feedstock materials, such dialkanolamines or trialkanolamines.

The quaternary ammonium ester compound may be derived from fatty acids characterized by an Iodine Value of from 0 to 140, or from 0 to about 90, or from about 10 to about 70, or from about 15 to about 50, or from about 18 to about 30. Iodine Values may be determined according to the method provided in US2020/0407665 (equivalent to WO2020/264566).

The composition may include a quaternary ammonium ester compound, a silicone, or combinations thereof, preferably a combination. The combined total amount of quaternary ammonium ester compound and silicone may be from about 5% to about 70%, or from about 6% to about 50%, or from about 7% to about 40%, or from about 10% to about 30%, or from about 15% to about 25%, by weight of the composition. The composition may include a quaternary ammonium ester compound and silicone in a weight ratio of from about 1:10 to about 10:1, or from about 1:5 to about 5:1, or from about 1:3 to about 1:3, or from about 1:2 to about 2:1, or about 1:1.5 to about 1.5:1, or about 1:1.

The composition may contain mixtures of different types of conditioning actives. The compositions of the present disclosure may contain a certain conditioning active but be substantially free of others. For example, the composition may be free of quaternary ammonium ester compounds, silicones, or both. The composition may comprise quaternary ammonium ester compounds but be substantially free of silicone. The composition may comprise silicone but be substantially free of quaternary ammonium ester compounds.

The conditioning active may comprise glyceride copolymers. The glyceride copolymers may be derived from natural oils. Examples of natural oils include, but are not limited to, vegetable oils, algae oils, fish oils, animal fats, tall oils, derivatives of these oils, combinations of any of these oils, and the like. Representative non-limiting examples of vegetable oils include low erucic acid rapeseed oil (canola oil), high erucic acid rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil, preferably canola oil. Representative non-limiting examples of animal fats include lard, tallow, poultry fat, yellow grease, and fish oil. Tall oils are by-products of wood pulp manufacture. The glyceride copolymers may be metathesized unsaturated polyol esters.

### C. Deposition Aid

The treatment compositions of the present disclosure may comprise a deposition aid. Deposition aids can facilitate deposition of the various benefit agents, including the esteramines or salts thereof of the present disclosure, conditioning actives, perfumes or perfume delivery systems (such as encapsulated perfumes), or combinations thereof, improving the performance benefits of the compositions and/or allowing for more efficient formulation of such benefit agents. The composition may comprise, by weight of the composition, from 0.0001% to 3%, preferably from 0.0005% to 2%, more preferably from 0.001% to 1%, or from about 0.01% to about 0.5%, or from about 0.05% to about 0.3%, of a deposition aid. The deposition aid may be a cationic or amphoteric polymer, preferably a cationic polymer.

Cationic polymers in general and their methods of manufacture are known in the literature. Suitable cationic polymers may include quaternary ammonium polymers known the “Polyquaternium” polymers, as designated by the International Nomenclature for Cosmetic Ingredients, such as Polyquaternium-6 (poly(diallyldimethylammonium chloride), Polyquaternium-7 (copolymer of acrylamide and diallyldimethylammonium chloride), Polyquaternium-10 (quaternized hydroxyethyl cellulose), Polyquaternium-22 (copolymer of acrylic acid and diallyldimethylammonium chloride), and the like.

The deposition aid may be selected from the group consisting of polyvinylformamide, partially hydroxylated polyvinylformamide, polyvinylamine, polyethylene imine, ethoxylated polyethylene imine, polyvinylalcohol, polyacrylates, and combinations thereof.

The cationic polymer may comprise a cationic acrylate and/or cationic methacrylate. Such polymers may be copolymers, for example further comprising a nonionic monomer, such as acrylamide. The cationic polymer may be linear or crosslinked. The deposition aid may comprise a combination of linear cationic polymers and crosslinked cationic polymers.

Deposition aids can be added concomitantly with delivery particles (at the same time with, e.g., encapsulated benefit agents, such as the encapsulated perfume) or directly/independently in the consumer product composition. The weight-average molecular weight of the polymer may be from 500 to 5000000 or from 1000 to 2000000 or from 2500 to 1500000 Dalton, as determined by size exclusion chromatography relative to polyethyleneoxide standards using Refractive Index (RI) detection. The weight-average molecular weight of the cationic polymer may be from 5000 to 37500 Dalton.

D. Additional Perfume and/or Perfume Delivery Systems

The treatment compositions of the present disclosure may comprise additional perfume and/or perfume delivery systems.

The treatment compositions of the present disclosure may comprise other perfume raw materials, for example in neat or free form, including PRMs that do not contain an aldehyde or ketone moiety. For example, other PRMs may be provided as neat or free oils to the treatment composition, even if they will not join with the esteramine or salt thereof. Such mixtures may be desirable, for example, to provide a more well-rounded olfactory experience.

The treatment compositions of the present disclosure may further comprise neat perfume, preferably neat perfume raw materials that does not comprise an aldehyde or ketone moiety. Preferably, the neat perfume comprises an alcohol-containing perfume raw material. Suitable alcohol-containing perfume raw materials are known to one of ordinary skill in the art, and may include geraniol, citronellol, cinnamic alcohol, eugenol, and the like. That being said, the neat perfume may further comprise free perfume raw materials that do comprise aldehyde and/or ketone moieties.

The treatment compositions of the present disclosure may, additionally or alternatively, comprise a perfume delivery system. Such perfume delivery systems may take the form of a polymer-assisted delivery system. Such perfume delivery systems may take the form of an encapsulate, for example a core-shell encapsulate, where the core comprises perfume raw materials and is surrounded by a polymeric shell. The polymeric shell may comprise polymeric material derived from polyacrylates, polyurea, polyurethanes, polysaccharides, polyvinyl alcohol, melamine, derivatives thereof, or combinations thereof. Additionally, or alternatively, suitable perfume delivery systems may include known pro-perfume/pro-fragrance materials.

### Other Materials

The treatment compositions, and/or even the premix compositions, of the present disclosure may comprise unreacted reactants and/or degradation products of the esteramine or salt thereof described herein. For example, the treatment compositions and/or premix compositions of the present disclosure may comprise: precursors or derivatives of the carbon-containing core alone, such as parent amino acids (e.g., H₂-A-H, where A is substantially as defined above according to Formula III, where G=oxygen); hydrophobically modified amino acids (e.g., H₂-A-Q, where A is substantially as defined above according to Formula III); free forms of the hydrophobe (e.g., H-G-Q, such as fatty alcohols like Dodecanol); forms of the esteramine or salt thereof that are free of the hydrophobe (e.g., Z-A-H, where A is substantially as defined above according to Formula III, where G=oxygen); free benefit agents, such as aldehyde- or ketone-containing PRMs; or combinations thereof.

### Method of Making a Treatment Composition

The present disclosure relates to processes for making any of the compositions described herein. The process of making a treatment composition, which may be a consumer product, may comprise the step of combining at least one perfume raw material and an esteramine or salt thereof as described herein with an adjunct material as described herein.

The at least one perfume raw material and/or esteramine or salt thereof compound may be combined with such adjunct materials by methods that include mixing and/or spraying.

The compositions of the present disclosure can be formulated into any suitable form and prepared by any process chosen by the formulator. The at least one perfume raw material, esteramine or salt thereof, and adjunct materials may be combined in a batch process, in a circulation loop process, and/or by an in-line mixing process. Suitable equipment for use in the processes disclosed herein may include continuous stirred tank reactors, homogenizers, turbine agitators, recirculating pumps, paddle mixers, high shear mixers, static mixers, plough shear mixers, ribbon blenders, vertical axis granulators and drum mixers, both in batch and, where available, in continuous process configurations, spray dryers, and extruders.

For example, the method of making a treatment composition may include the step of combining at least one perfume raw material and/or an esteramine or salt thereof according to the present disclosure with a base composition, preferably a liquid base composition, where the base composition comprises an adjunct ingredient. This process may occur, for example, in a batch process or in an in-line mixing process, preferably an in-line mixing process.

The method of making a treatment composition may include the step of combining at least one perfume raw material, and/or an esteramine or salt thereof, a benefit agent, and an adjunct ingredient, as described herein. Preferably, the adjunct ingredient is part of a base composition, and at least one perfume raw material, esteramine or salt thereof, and/or the benefit agent are each added to the base composition as separate inputs. The separate inputs may be added sequentially (e.g. in series), or substantially simultaneously. Preferably, the base composition is a liquid. This process may occur, for example, in a batch process or in an in-line mixing process, preferably an in-line mixing process.

The method of making a treatment composition may include the step of adding a premix to a base composition. The premix composition may comprise at least one perfume raw material, an esteramine or salt thereof, and/or a benefit agent, as described herein. The premix composition may be obtainable by combining at least one perfume raw material, and/or an esteramine or salt thereof with a benefit agent, as described herein. The premix composition may include at least one perfume raw material, and an esteramine or salt thereof according to the present disclosure, for example due to the reaction of the esteramine or salt thereof and the benefit agent. The premix may optionally contain water. This process may occur, for example, in a batch process or in an in-line mixing process, preferably an in-line mixing process. A premix may be particularly preferred when making a treatment composition that is in, or will be in solid form, such as a PEG-based pastille. In such cases, removal or reduction of water from the premix may be useful, for example via a water scavenger such as magnesium sulfate, or via the use of a molecular sieve or distilled off in vacuo.

### Method of Treating a Surface

The present disclosure further relates to methods of treating a surface (for example, a surface of an article) with a treatment composition according to the present disclosure. Such methods may provide cleaning, conditioning, hygienic, and/or freshening benefits.

Suitable surfaces may include fabrics (including clothing, towels, or linens), hard surfaces (such as tile, porcelain, linoleum or wood floors), dishware, hair, skin, or mixtures thereof.

The method may include a step of contacting an article or surface with a treatment composition of the present disclosure, optionally in the presence of water, optionally further including the step of rinsing and/or drying the article or surface. The treatment composition may be in neat form or diluted in a liquor, for example, a wash or rinse liquor. The treatment composition may be diluted in water prior, during, or after contacting the surface or article. The surface, or an article comprising such a surface, may be optionally washed and/or rinsed before and/or after the contacting step.

The method of treating and/or cleaning a surface may include the steps of:
a) optionally washing, rinsing and/or drying the surface;
b) contacting the surface with a treatment composition as described herein, optionally in the presence of water;
c) optionally washing and/or rinsing the surface; and
d) optionally drying the surface by drying passively and/or via an active method such as a laundry dryer.

For purposes of the present disclosure, washing includes but is not limited to, scrubbing, and mechanical agitation. The fabric may comprise most any fabric capable of being laundered or treated in normal consumer or industrial use conditions.

Liquors that may comprise the disclosed compositions may have a pH of from about 3 to about 11.5. When diluted, such compositions are typically employed at concentrations of from about 500 ppm to about 15,000 ppm in solution. When the wash solvent is water, the water temperature typically ranges from about 5° C. to about 90° C. and, when the surface is part of a fabric, the water to fabric weight ratio is typically from about 1:1 to about 30:1.

The present disclosure further discloses a process of treating a surface or article, preferably a fabric, with an aqueous treatment liquor that comprises an orthoester, at least one perfume raw material, and an esteramine or salt thereof according to the present disclosure. The process may include the step of contacting the surface or article, preferably a fabric, with the aqueous liquor. The esteramine or salt thereof may be present in the aqueous liquor at a level of from about 0.001 ppm (e.g., 1 ppb) to about 1000 ppm by weight.

The present disclosure further discloses a process of treating a surface or article, preferably a fabric, with an aqueous treatment liquor that comprises the orthoester, at least one perfume raw material, and/or an esteramine or salt thereof according to the present disclosure. The process may include the step of contacting the surface or article, preferably a fabric, with the aqueous liquor. The esteramine or salt thereof may be present in the aqueous liquor at a level of from about. 001 ppm (e.g., 1 ppb) to about 1000 ppm by weight.

### Use

The present disclosure relates to the use of the presently described treatment compositions for providing a freshness benefit.

The present disclosure relates to the use of the presently described treatment compositions for providing an anti-malodor benefit.

### Combinations

Specifically contemplated combinations of the disclosure are herein described in the following lettered paragraphs. These combinations are intended to be illustrative in nature and are not intended to be limiting.
A. A treatment composition comprising at least one perfume raw material and an esteramine of Formula (I) or salt thereof,

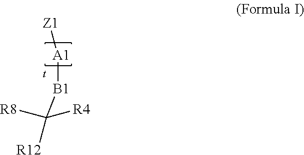

wherein independently from each other:
t being an integer from 1 to 100;
A₁ is independently for each repetition unit t selected from the list consisting of ethyleneoxy group, 1,2-propyleneoxy group, 1,2-butyleneoxy group, 2,3-butyleneoxy group, i-butyleneoxy group, pentyleneoxy group, hexyleneoxy group, styryloxy group, decenyloxy group, dodecenyloxy group, tetradecenyloxy group, and hexadecanyloxy group, wherein for t equal to 1 the oxygen atom of the A₁ group is bound to the B group and the following A₁ group is always bound via the oxygen atom to the previous A₁ group;
B₁ is independently from each other selected from the group consisting of a bond, linear C₁ to C₁₂ alkanediyl groups, and branched C₁ to C₁₂ alkanediyl groups;
R₄, R₈, and R₁₂ being selected from the group consisting of H, linear alkyl, branched alkyl, and cycloalkyl;
with the provisio that Z₁ is selected from the group consisting of alanine, arginine, asparagine, aspartic acid, cysteine, glutamine, glycine, histidine, isoleucine, leucine, lysine, methionine, phenylalanine, proline, serine, threonine, tryptophan, tyrosine, valine, and a compound according to Formula (II), wherein said compound according to Formula (II) connects to the compound according to Formula (I) via the bond labeled with *, with the provisio of at least one group R₄, R₈, and/or R₁₂ containing at least 7 or more carbon atoms;

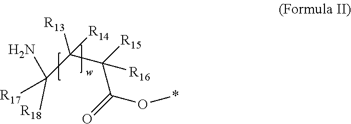

with independently from each other
w being an integer from 0 to 12;
R₁₃ and R₁₄ independently for each repetition unit w being selected from the group consisting of H, linear alkyl, branched alkyl, and cycloalkyl;
R₁₅, R₁₆, R₁₇, and R₁₈ being selected from the group consisting of H, linear alkyl, branched alkyl, and cycloalkyl.
B. The treatment composition according to paragraph A, comprising the salt of the esteramine, wherein the salt is formed by at least partial protonation of the amine group by an acid being a protic organic or inorganic acid.
C. The treatment composition according to any of paragraphs A-B, wherein the salt is formed by at least partial protonation of the amine group by an acid being selected from the group consisting of methanesulfonic acid, hydrochloric acid, hydrobromic acid, sulfuric acid, phosphoric acid, citric acid and lactic acid.
D. The treatment composition according to any of paragraphs A-C, wherein A₁ is independently for each repetition unit t selected from the list consisting of ethyleneoxy group, 1,2-propyleneoxy group, and 1,2-butyleneoxy group.
E. The treatment composition according to any of paragraphs A-D, wherein Z₁ is selected from the group consisting of alanine, glycine, lysine, and a compound according to Formula (II), wherein w is an integer in the range of from 1 to 4, and wherein the compound according to Formula (II) connects to the compound according to Formula (I) via the bond labeled with *, with the provisio of at least one group R₄, R₈, and/or R₁₂ containing at least 7 or more carbon atoms.
F. A treatment composition comprising at least one perfume raw material and an esteramine or salt thereof, the esteramine or salt thereof obtained by a process of using catalytic amounts of at least one orthoester, the process comprising the steps of: a) reaction of: i) at least one amino acid selected from alpha-, beta-, gamma-, delta-, epsilon-etc. amino acids, such as alanine, glycine, leucine, isoleucine, valine, proline, phenylalanine, arginine, asparagine, aspartic acid, aspartate, glutamine, glutamate, histidine, lysine, threonine, tryptophan, tyrosine, cysteine, methionine, serine; alpha-amino acids with secondary or tertiary amino groups such as sarcosine, N,N-dimethylglycine; other amino acids such as 6-aminohexanoic acid, 4-aminobutanoic acid, 3-aminopropanoic acid, 12-aminododecanoic acid, 11-aminoundecanoic acid; amino acids formally derived from the hydrolysis of α-lactam (three ring atoms), β-lactam (four ring atoms), γ-lactam (five ring atoms) and so on; such lactams preferably being β-propiolactam, g-butyrolactam, δ-valerolactam, g-valerolactam, e-caprolactam, d-decalactam, g-decalactam, e-decalactam; preferably alanine, valine, beta-alanine, 6-aminohexanoic acid; with ii) at least one alcohol (A) bearing at least one hydroxy group, being selected from mono-, di- and polyols, all of which may be optionally alkoxylated, wherein the alkoxylation of the at least one hydroxy group takes place in a step before step a), with the alcohol being alkoxylated with at least one alkylene oxide, preferably at least 1 and up to 200, preferably 1 to 100, more preferably up to 50 moles alkylene oxide per hydroxy group; in presence of iii) at least one acid (C), being selected from inorganic and organic acids, wherein said organic or inorganic acid has preferably a pKa value in the range of from −3 and up to +5, more preferably from −2.5 to 1.5, preferably at least one organic acid, such as sulfonic acids, more preferably alkylsulfonic acid and/or arylsulfonic acid; and iv) in the presence of at least one orthoester, such as triethyl orthoformate, trimethyl orthoformate, triethyl orthoacetate, trimethyl orthoacetate, and the like; whereas the orthoester is used in sub-stoichiometric amounts, preferably catalytic amounts (referred to the amino acid); and whereas the alcohol used for the esterification is different from the alcohol-residual in the orthoester; to produce an esteramine salt; b) optional neutralization of the obtained esteramine salt with at least one base to obtain the free esteramine.
G. The treatment composition according to paragraph F, wherein the alcohol (A) is selected from: a. mono-alcohols such as C1- to C36-alkanols, selected from the groups non-alkoxylated linear C2- to C36-alcohols, such as mixture of such alcohols selected from C6-to C22-fatty alcohols, preferably C8- to C22-fatty alcohols, more preferably C12- and C14-fatty alcohols, most preferably C16- and C18-fatty alcohols; non-alkoxylated branched C3- to C36-alcohols such as 2-ethylhexanol, 2-propylheptanol, isotridecanol, isononanol, C9-C17 oxoalcohols; alkoxylated linear C2- to C36-alcohols such as alkoxylated mixture of C6- to C22-fatty alcohols, preferably alkoxylated mixtures of C8- to C22-fatty alcohols, more preferably alkoxylated mixtures of C12- and C14-fatty alcohols, most preferably alkoxylated mixtures of C16- and C18-fatty alcohols; alkoxylated branched C3- to C36-alcohols such as alkoxylated 2-ethylhexanol, alkoxylated 2-propylheptanol, alkoxylated isotridecanol, alkoxylated isononanol, alkoxylated C9-C17 oxoalcohols; b. di-alcohols such as alkane diols, polyalkoxylated C2-C6-alkanediols bearing at least two hydroxy groups, c. oligo-alcohols such as polyalkoxylated C3-C6-alkanetriols, bearing at least three hydroxy groups, d. polyols such as sugar alcohols, polyalkoxylated C5-C6-alkane polyols, glycerols such as diglycerol, triglycerol polyglycerol, dipentaerythritol, tripentaerythritol; and/or e. phenoxyalkanols such as phenoxyethanol; with the alcohol(s) selected from the groups of mono-alcohols and alkoxylated di-, oligo-alcohols and alkoxylated polyols being preferred, and the alcohols selected from the group(s) mono-alcohols and alkoxylated di-alcohols being even more preferred.
H. The treatment composition according to any of paragraphs F-G, wherein the alcohol (A) employed is an alkoxylated alcohol which is obtained by alkoxylating at least one hydroxy group of the alcohol according to Claim 2 with one or more alkylene oxides to produce alkylene oxy-chains comprising one or more moieties stemming from alkylene oxides selected from C2 to C22-alkylene oxides, preferably C2-C4-alkylene oxides, whereas the moieties stemming from the alkylene oxide(s) may be arranged in random, block or multiblock-order or combinations thereof, preferably as block, more preferably contains only one block consisting of ethylene oxide or consisting of two blocks with the first block—preferably the “inner block” directly linked to the hydroxy group of the alcohol—consisting of ethylene oxide and a second block—preferably being the “outer block linked to the ethylene oxide-block—consisting of propylene oxide, such di-blcok even more preferably consisting of 3 to 10 EO-derived moieties and the PO-block consisting of 1 to 10 PO-derived moieties.
I. The treatment composition according to any of paragraphs F-H, wherein the acid (C) is selected from: i) alkyl sulfonic acids, such as methanesulfonic acid, ethylsulfonic acid, propylsulfonic acid, camphorsulfonic acid; alkylarylsulfonic acids and specifically alkylbenzenesulfonic acids, such as toluenesulfonic acid (including the mixture of isomers thereof), p-toluenesulfonic acid, o-toluenesulfonic acid, m-toluenesulfonic acid, xylenesulfonic acid (mixture of isomers), 2, 6-dimethylbenzenesulfonic acid, 2,5-dimethylbenzenesulfonic acid, 2, 4-dimethylbenzenesulfonic acid, 4-dodecylbenzenesulfonic acid, iso-propyl benzenesulfonic acid, ethylbenzenesulfonic acid, and naphthalenesulfonic acid, preferably p-toluenesulfonic acid and methanesulfonic acid, more preferably methanesulfonic acid;
ii) inorganic acids such as hydrochloric acid, hydrobromic acid, phosphoric acid;
preferably selected from group i).
J. The treatment composition according to any of paragraph F-I, wherein the acid (C) is chosen such that the esteramine is obtained as salt in cationic form, preferably the acid chosen is methanesulfonic acid and the esteramine obtained is a salt in cationic form.
K. The treatment composition according to any of paragraphs F-J, wherein the molar ratio of amino acid to hydroxyl group of the (optionally alkoxylated) alcohol is (0.8*n):1 to (1*n):1.5, with the number of hydroxy groups of the (optionally alkoxylated) alcohol being n.
L. The treatment composition according to any of paragraphs F-K, wherein the process is carried out with the molar ratio of the acid (C) to the amino acid is in the range of from 0.8:1 to 1:1.2.
M. The treatment composition according to any of paragraphs F-L, wherein in the process the reaction is performed: a. at a temperature of from 50 to 200° C., preferably 70-180° C., more preferably 80° C.-160° C., most preferably 120° C.-150° C., such as 60, 65, 75, 85, 90, 95, 100, 110, 115, 120, 125, 130, 135, 140, 145, 155, 165, 170, 190° C.; b. for a period of from 1 to 30, preferably from 2, more preferably from 3 hours, even more preferably at least 5 hours, and preferably up to 48, more preferably up to 20, even more preferably up to 15 hours, such as preferably 3 to 24, more preferably 5 to 24, most preferably 10-24 hour(s); and c. at from 0.001 to 10 bar pressure, such as from 0.001, more preferably from 0,005, even more preferably from 0.1, and preferably up to 8, more preferably up to 5, even more preferably up to 4 bar, such as 1 to 10, more preferably 1 to 5, even more preferably 1 to 4 bar, or such as 1 to 1000 mbar, more preferably 100 to 500 mbar.
N. The treatment composition according to any of paragraphs F-M, wherein in the process the solvents for the reaction are selected from water, toluene, xylene, heptanol, cyclohexene, and the like, preferably only being water.
O. The treatment composition according to any of paragraphs F-N, wherein during or following the reaction, preferably at least during the reaction, water and/or excess alcohol are removed, such removal preferably being carried out by application of a stream of gas such as using gas such as inert gas as nitrogen or argon, preferably nitrogen, or steam made from water, preferably using inert gas, more preferably nitrogen, and/or applying a distillation method, preferably a distillation, more preferably a distillation method under reduced pressure and/or at elevated temperature, preferably both, a more preferred method being the use of an apparatus such as a Dean-Stark-trap, most preferably using a Dean-Stark-trap, such removal more preferably carried out applying a vacuum in the range of from 0.1 mbar to 800 mbar, preferably of from 1 mbar to 500 mbar and more preferably of from 10 mbar to 100 mbar, and using elevated temperatures.
P. The treatment composition according to any of paragraphs A-O, wherein the treatment composition is a consumer product, preferably a consumer product selected from a fabric care composition, a hard surface cleaner composition, a dish care composition, a hair care composition, a body cleansing composition, or a mixture thereof.
Q. The treatment composition according to any of paragraphs A-P, wherein the treatment composition further comprises a benefit agent.
R. The treatment composition according to any of paragraphs A-Q, wherein the benefit agent is selected from an antimicrobial agent, a pesticide, an insect repellant, an anti-fungal agent, a herbicidal agent, a hueing dye, an antioxidant, a non-perfume organoleptic, or a combination thereof.
S. The treatment composition according to any of paragraphs A-R, wherein the at least one perfume raw material is selected from the group consisting of: methyl nonyl acetaldehyde: benzaldehyde; floralozone; isocyclocitral; triplal (ligustral); precylcemone B; lilial; decyl aldehyde; undecylenic aldehyde; cyclamen homoaldehyde; cyclamen aldehyde; dupical; oncidal; adoxal; melonal; calypsone; anisic aldehyde; heliotropin; cuminic aldehyde; scentenal; 3,6-dimethylcyclohex-3-ene-1-carbaldehyde; satinaldehyde; canthoxal; vanillin; ethyl vanillin; cinnamic aldehyde; cis-4-decenal; trans-4-decenal; cis-7-decenal; undecylenic aldehyde; trans-2-hexenal; trans-2-octenal; 2-undecenal; 2,4-dodecadeienal; cis-4-heptenal; Florydral; butyl cinnamaldehyde; limonelal; amyl cinnamaldehyde; hexyl cinnamaldehyde; citronellal; citral; cis-3-hexen-1-al; nerolione; 4-(4-methoxyphenyl) butan-2-one; 1-naphthalen-2-ylethanone; nectaryl; trimofix O; fleuramone; delta-damascone; beta-damascone; alpha-damascone; methyl ionone; 2-hexylcyclopent-2-en-1-one; galbascone; and mixtures thereof; more preferably wherein the hydrophobic moiety is an organic group comprising from about 8 to about 18 chain atoms, preferably wherein the chain atoms are carbon atoms.
T. The treatment composition according to any of paragraphs A-S, wherein the at least one perfume raw material comprises an aldehyde moiety.
U. The treatment composition according to any of paragraphs A-T, wherein the treatment composition further comprises an adjunct ingredient comprising one or more of the following: surfactants, conditioning actives, deposition aids, rheology modifiers or structurants, antioxidants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, perfume delivery systems, structure elasticizing agents, carriers, hydrotropes, processing aids, anti-agglomeration agents, coatings, formaldehyde scavengers, and/or pigments.
V. The treatment composition according to any of paragraphs A-U, wherein the adjunct ingredient comprises a conditioning active, preferably wherein the conditioning active comprises quaternary ammonium ester compounds, more preferably wherein the quaternary ammonium ester compounds are present at a level of from about 2 wt % to about 35 wt %, preferably from about 4 wt % to about 25 wt %, more preferably from about 5 wt % to about 20 wt %, preferably from about 6 wt % to about 15 wt %, more preferably from about 7 wt % to about 12 wt %, by weight of the treatment composition.
W. The treatment composition according to any of paragraphs A-V, wherein the treatment composition is in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam or mousse, a non-woven sheet, or a mixture thereof, preferably a liquid composition.
X. The treatment composition according to any of paragraphs A-W, wherein the esteramine or salt thereof is present in the treatment composition at a level of from about 0.001% to about 30%, by weight of the treatment composition, more preferably about 0.01% to about 5%, more preferably about 0.1% to about 3%, even more preferably about 0.5% to about 2%.
Y. The treatment composition according to any of paragraphs A-X, wherein the at least one perfume raw material is present in the treatment composition at a level of from about 0.001% to about 30%, by weight of the treatment composition, more preferably about 0.01% to about 5%, more preferably about 0.1% to about 3%, even more preferably about 0.5% to about 2%.
Z. A method of treating an article or a surface, wherein the method comprises treating the article or surface with the treatment composition according to any of paragraphs A-Y, optionally in the presence of water.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this 5 invention.

What is claimed is:

1. A treatment composition comprising at least one perfume raw material and an esteramine of Formula (I) or salt thereof, 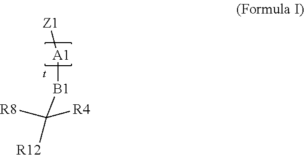
wherein independently from each other:
t being an integer from 1 to 100;
A₁ is independently for each repetition unit t selected from the list consisting of ethyleneoxy group, 1,2-propyleneoxy group, 1,2-butyleneoxy group, 2,3-butyleneoxy group, i-butyleneoxy group, pentyleneoxy group, hexyleneoxy group, styryloxy group, decenyloxy group, dodecenyloxy group, tetradecenyloxy group, and hexadecanyloxy group, wherein for t equal to 1 the oxygen atom of the A₁ group is bound to the B group and the following A₁ group is always bound via the oxygen atom to the previous A₁ group;
B₁ is independently from each other selected from the group consisting of a bond, linear C₁ to C₁₂ alkanediyl groups, and branched C₁ to C₁₂ alkanediyl groups;
R₄, R₈, and R₁₂ being selected from the group consisting of H, linear alkyl, branched alkyl, and cycloalkyl;
with the provisio that Z₁ is selected from the group consisting of alanine, arginine, asparagine, aspartic acid, cysteine, glutamine, glycine, histidine, isoleucine, leucine, lysine, methionine, phenylalanine, proline, serine, threonine, tryptophan, tyrosine, valine, and a compound according to Formula (II), wherein said compound according to Formula (II) connects to the compound according to Formula (I) via the bond labeled with *, with the provisio of at least one group R₄, R₈, and/or R₁₂ containing at least 7 or more carbon atoms; 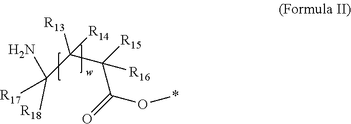
with independently from each other
w being an integer from 0 to 12;
R₁₃ and R₁₄ independently for each repetition unit w being selected from the group consisting of H, linear alkyl, branched alkyl, and cycloalkyl;
R₁₅, R₁₆, R₁₇, and R₁₈ being selected from the group consisting of H, linear alkyl, branched alkyl, and cycloalkyl.

2. The treatment composition of claim 1, comprising the salt of the esteramine, wherein the salt is formed by at least partial protonation of the amine group by an acid being a protic organic or inorganic acid.

3. The treatment composition of claim 2, wherein the salt is formed by at least partial protonation of the amine group by an acid being selected from the group consisting of methanesulfonic acid, hydrochloric acid, hydrobromic acid, sulfuric acid, phosphoric acid, citric acid and lactic acid.

4. The treatment composition according to claim 1, wherein A₁ is independently for each repetition unit selected from the list consisting of ethyleneoxy group, 1,2-propyleneoxy group, and 1,2-butyleneoxy group.

5. The treatment composition according claim 1, wherein Z₁ is selected from the group consisting of alanine, glycine, lysine, and a compound according to Formula (II), wherein w is an integer in the range of from 1 to 4, and wherein the compound according to Formula (II) connects to the compound according to Formula (I) via the bond labeled with *, with the provisio of at least one group R₄, R₈, and/or R₁₂ containing at least 7 or more carbon atoms.

6. A treatment composition comprising at least one perfume raw material and an esteramine or salt thereof, the esteramine or salt thereof obtained by a process of using catalytic amounts of at least one orthoester, the process comprising the steps of:
a) reaction of:
i) at least one amino acid selected from alpha-, beta-, gamma-, delta-, epsilon-etc. amino acids, such as alanine, glycine, leucine, isoleucine, valine, proline, phenylalanine, arginine, asparagine, aspartic acid, aspartate, glutamine, glutamate, histidine, lysine, threonine, tryptophan, tyrosine, cysteine, methionine, serine; alpha-amino acids with secondary or tertiary amino groups such as sarcosine, N,N-dimethylglycine; other amino acids such as 6-aminohexanoic acid, 4-aminobutanoic acid, 3-aminopropanoic acid, 12-aminododecanoic acid, 11-aminoundecanoic acid; amino acids formally derived from the hydrolysis of α-lactam (three ring atoms), β-lactam (four ring atoms), γ-lactam (five ring atoms) and so on; such lactams preferably being β-propiolactam, g-butyrolactam, δ-valerolactam, g-valerolactam, e-caprolactam, d-decalactam, g-decalactam, e-decalactam; preferably alanine, valine, beta-alanine, 6-amino hexanoic acid;
with
ii) at least one alcohol (A) bearing at least one hydroxy group, being selected from mono-, di- and polyols, all of which may be optionally alkoxylated, wherein the alkoxylation of the at least one hydroxy group takes place in a step before step a), with the alcohol being alkoxylated with at least one alkylene oxide, preferably at least 1 and up to 200, preferably 1 to 100, more preferably up to 50 moles alkylene oxide per hydroxy group;
in presence of
iii) at least one acid (C), being selected from inorganic and organic acids, wherein said organic or inorganic acid has preferably a pKa value in the range of from −3 and up to +5, more preferably from −2.5 to 1.5, preferably at least one organic acid, such as sulfonic acids, more preferably alkylsulfonic acid and/or arylsulfonic acid;
and
iv) in the presence of at least one orthoester, such as triethyl orthoformate, trimethyl orthoformate, triethyl orthoacetate, trimethyl orthoacetate, and the like;
whereas the orthoester is used in sub-stoichiometric amounts, preferably catalytic amounts (referred to the amino acid); and
whereas the alcohol used for the esterification is different from the alcohol-residual in the orthoester;
to produce an esteramine salt;
b) optional neutralization of the obtained esteramine salt with at least one base to obtain the free esteramine;
wherein the treatment composition comprises, by weight of the total composition, less than 0.5% of alcohol (A) as a residual of the reaction process which was used to obtain the esteramine or salt thereof.

7. The treatment composition according to claim 6, wherein the alcohol (A) is selected from:
a. mono-alcohols such as C1- to C36-alkanols, selected from the groups non-alkoxylated linear C2- to C36-alcohols; alkoxylated linear C2- to C36-alcohols; and
alkoxylated branched C3- to C36-alcohols;
b. di-alcohols such as alkane diols, polyalkoxylated C2-C6-alkanediols bearing at least two hydroxy groups,
c. oligo-alcohols such as polyalkoxylated C3-C6-alkanetriols, bearing at least three hydroxy groups,
d. polyols such as sugar alcohols, polyalkoxylated C5-C6-alkane polyols, glycerols such as diglycerol, triglycerol polyglycerol, dipentaerythritol, tripentaerythritol; and/or
e. phenoxyalkanols such as phenoxyethanol;
with the alcohol(s) selected from the groups of mono-alcohols and alkoxylated di-, oligo-alcohols and alkoxylated polyols.

8. The treatment composition according to claim 6, wherein the acid (C) is selected from:
i) alkyl sulfonic acids, such as methanesulfonic acid, ethylsulfonic acid, propylsulfonic acid, camphorsulfonic acid; alkylarylsulfonic acids and alkylbenzenesulfonic acids, such as toluenesulfonic acid (including the mixture of isomers thereof), p-toluenesulfonic acid, o-toluenesulfonic acid, m-toluenesulfonic acid, xylenesulfonic acid (mixture of isomers), 2, 6-dimethylbenzenesulfonic acid, 2,5-dimethylbenzenesulfonic acid, 2,4-dimethylbenzenesulfonic acid, 4-dodecylbenzenesulfonic acid, iso-propyl benzenesulfonic acid, ethylbenzenesulfonic acid, and naphthalenesulfonic acid, preferably p-toluenesulfonic acid and methanesulfonic acid;
ii) inorganic acids such as hydrochloric acid, hydrobromic acid, phosphoric acid.

9. The treatment composition according to claim 6, wherein the acid (C) is chosen such that the esteramine is obtained as a salt in cationic form.

10. The treatment composition according to claim 6, wherein the molar ratio of amino acid to hydroxyl group of the alcohol is (0.8*n):1 to (1*n):1.5, with the number of hydroxy groups of the alcohol being n.

11. The treatment composition according to claim 6, wherein the process is carried out with the molar ratio of the acid (C) to the amino acid is in the range of from 0.8:1 to 1:1.2.

12. The treatment composition according to claim 6, wherein in the process the reaction is performed:
a. at a temperature of from 50 to 200° C., preferably 70-180° C.;
b. for a period of from 1 to 30 hour(s); and
c. at from 0.001 to 10 bar pressure.

13. The treatment composition according to claim 6, wherein in the process the solvents for the reaction are selected from water, toluene, xylene, heptanol, and cyclohexene.

14. The treatment composition according to claim 6, wherein during or following the reaction, water and/or excess alcohol are removed.

15. The treatment composition according to claim 6, wherein the treatment composition is a consumer product.

16. The treatment composition according to claim 6, wherein the treatment composition further comprises a benefit agent.

17. The treatment composition according to claim 6, wherein the benefit agent is selected from an antimicrobial agent, a pesticide, an insect repellant, an anti-fungal agent, a herbicidal agent, a hueing dye, an antioxidant, a non-perfume organoleptic, or a combination thereof.

18. The treatment composition according to claim 6, wherein the at least one perfume raw material is selected from the group consisting of: methyl nonyl acetaldehyde: benzaldehyde; floralozone; isocyclocitral; triplal (ligustral); precylcemone B; lilial; decyl aldehyde; undecylenic aldehyde; cyclamen homoaldehyde; cyclamen aldehyde; dupical; oncidal; adoxal; melonal; calypsone; anisic aldehyde; heliotropin; cuminic aldehyde; scentenal; 3,6-dimethylcyclohex-3-ene-1-carbaldehyde; satinaldehyde; canthoxal; vanillin; ethyl vanillin; cinnamic aldehyde; cis-4-decenal; trans-4-decenal; cis-7-decenal; undecylenic aldehyde; trans-2-hexenal; trans-2-octenal; 2-undecenal; 2,4-dodecadeienal; cis-4-heptenal; Florydral; butyl cinnamaldehyde; limonelal; amyl cinnamaldehyde; hexyl cinnamaldehyde; citronellal; citral; cis-3-hexen-1-al; nerolione; 4-(4-methoxyphenyl) butan-2-one; 1-naphthalen-2-ylethanone; nectaryl; trimofix O; fleuramone; delta-damascone; beta-damascone; alpha-damascone; methyl ionone; 2-hexylcyclopent-2-en-1-one; galbascone; and mixtures thereof.

19. The treatment composition according to claim 19, wherein the at least one perfume raw material comprises an aldehyde moiety.
